(19) United States
(12) Patent Application Publication
(10) Pub. No.: US 2015/0216888 A1
(43) Pub. Date: Aug. 6, 2015

(54) ANTIPARASITIC POLYANHYDRIDE NANOPARTICLES

(71) Applicant: Iowa State University Research Foundation, Inc., Ames, IA (US)

(72) Inventors: Bryan Howard Bellaire, Ankeny, IA (US); Balaji Narasimhan, Ames, IA (US)

(73) Assignee: Iowa State University Research Foundation, Inc., Ames, IA (US)

(21) Appl. No.: 14/614,333

(22) Filed: Feb. 4, 2015

Related U.S. Application Data
(60) Provisional application No. 61/935,813, filed on Feb. 4, 2014.
(60) Provisional application No. 61/936,074, filed on Feb. 5, 2014.

(51) Int. Cl. A61K 31/7048; A61K 31/4184; A61K 31/495; A61K 9/51; A61K 31/65

(52) U.S. Cl. CPC A61K 31/7048; A61K 9/51; A61K 31/65; A61K 31/495; A61K 31/4184

(57) ABSTRACT

Filarial parasites Brugia, Wuchereria, Loa Loa and Onchocerca cause over 20 million infections worldwide and pose a significant social and economic burden in endemic areas. The invention provides compositions and methods to treat parasitic infections in animals and plants, and to kill and inhibit the replication of parasites in infected hosts. The methods can include administering to a host in need of treatment an effective antiparasitic amount of a composition comprising biodegradable polyanhydride microparticles or nanoparticles that encapsulate antiparasitic agents, optionally in combination with antibacterial agents. Through co-encapsulation of antiparasitic and antibacterial agents into the particles, the invention provides the ability to effectively kill parasitic helminthes, worms, and flukes, with up to a 40-fold reduction in the amount of drug used. The results described herein demonstrate the effectiveness of the drug carriers to reduce both the course of treatment and the amount of drug needed to treat parasitic infections.

### RELATED APPLICATIONS

This application claims priority under 35 U.S.C. §119(e) to U.S. Provisional Patent Application No. 61/935,813, filed Feb. 4, 2014, and 61/936,074, filed Feb. 5, 2014, which applications are incorporated herein by reference.

### BACKGROUND

Filarial parasites Brugia, Wuchereria, Loa Loa and Onchocerca are roundworms that exhibit complex life cycles requiring an arthropod host for larval stage development and subsequent transmission into humans. Over 140 million people live in areas endemic to filarial parasites. Improvements in controlling transmission and improving therapeutic interventions is an ever present pursuit for these neglected tropical diseases. Compounding difficulties in improving therapies are global, social, and economic limitations that effect distribution, cost and patient compliance. In 2000, the WHO initiated a call to eliminate lymphatic filariasis as a public-health problem by 2020. Significant progress has been made toward this eradication effort but the major steps have been limited to use of mass drug administration of a microfilaricide to interrupt transmission and diminish morbidity. This approach over the last 13 years has only reduced the reoccurrence in some countries by 46%. Drugs with the greatest efficacy toward lymphatic filariasis have been limited to trials of annual, bi-annual, single-dose, multiple dose, and combinations of either ivermectin (IVM) or diethylcarbamazine (DEC) with albendazole (ABZ).

In the past decade, the discovery of an endosymbiotic bacterium, Wolbachia has provided an additional target for therapies because killing the bacterium leads to the slow, but eventual death of the adult worm. Recently, antimicrobial treatments such as doxycycline have been added to antifilarial regimens to target Wolbachia. Anti-Wolbachia drugs have been shown to reduce pathogenicity and reproductive capacity of adult filarial worms. The limiting factors of the above drugs has been threefold, including the limitation of age restriction with some drugs due to cytotoxicity, the inability to reach the deep tissues where the adult filarial nematode resides, and patience drug compliance. These limiting factors have created the need to improve and expand therapy protocols.

Diseases such as river blindness (onchocerciasis) and lymphatic filariasis (elephantiasis) cause considerable suffering and pain in human populations living in tropical areas. These diseases are transmitted through biting insects that deposit parasite larvae into host tissues that develop into adult worms that shed offspring. The ability to treat parasitic infections is greatly limited by the solubility and absorption of drugs by the parasite. Accordingly, new methods and antiparasitic formulations are needed for the delivery of drugs to parasites. Formulations that can target and/or deliver antiparasitic agents to the intracellular environment of cells infected with parasites would be a significant benefit to large populations in tropical areas. Antiparasitic formulations that provide enhanced activity compared to administration of a corresponding free drug are urgently needed.

### SUMMARY

Amphiphilic polyanhydride nanoparticles (PANs) are chemically and structurally distinct from other polymer or lipid based particle delivery systems. PANs are solid, surface eroding particles that can encapsulate small molecules or proteins within the polymer matrix, providing the sustained release of drug as the PAN erodes within a target parasite. Antiparasitic and antimicrobial compounds can be encapsulated into PANs, thereby allowing the compounds to be slowly released after they are internalized by parasites as the particles degrade (FIG. 1). This next generation platform thus has the capability to be a multiple drug delivery system with the ability to increase the efficacy of the drug and parasite interaction. The ability of the PANs to slowly erode and release the cargo molecules in a controlled manner allows for specificity against both adult nematodes and the symbiotic bacteria Wolbachia. Administration of the PANs can therefore interrupt the life cycle of the nematode not by just reducing microfilaria load, but by directly increasing mortality in the adult population.

The invention provides for the use of a polyanhydride nanoparticle-based platform for the co-delivery of antibiotics and antiparasitic agents. In one embodiment, the invention provides PANs that deliver doxycycline to the symbiotic bacteria, Wolbachia, and that deliver the antiparasitic drug ivermectin to reduce microfilarial burden. The enhanced ability to co-deliver doxycycline and ivermectin in polyanhydride nanoparticles (PANs) to effectively kill adult female B. malayi filarial worms with up to a 40-fold reduction in the amount of drug used is described herein. Additionally, the time to death of the macrofilaria was significantly reduced when the antifilarial drug cocktail was delivered by PANs. The mechanism behind this enhanced killing of the macrofilaria can include the ability of the PANs to penetrate the outer membrane of parasites to effectively deliver drugs directly to the parasite and its symbiotic bacteria Wolbachia, at high enough microenvironment concentrations to cause death. Use of these methods can provide a significant reduction in the amount of drug required and the length of treatment required for treating filarial infections.

Accordingly, the invention provides a method to kill a parasite or inhibit the preproduction of parasites comprising: contacting a parasite with, or administering to the host of a parasite, an effective amount of a composition comprising polyanhydride nanoparticles. The polyanhydride nanoparticles can comprise: (a) polyanhydride polymers in the form of a nanoparticle and (b) a combination of two or more different active agents located in the interior of the nanoparticle, wherein the nanoparticle is substantially spherical in shape and has an average diameter of about 100 nm to about 900 nm. The polyanhydride polymers can include anhydride copolymers of 1,ω-bis(carboxy)(C₂-C₁₀)alkane units and 1,ω-bis(carboxyphenoxy)(C₂-C₁₀)alkane units. One active agent can be an antiparasitic agent and a second active agent can be an antibiotic agent. The nanoparticles can degrade by surface erosion in the presence of the parasite over a period of time to release the active agents from the interior of the nanoparticles, thereby killing the parasite or inhibiting the reproduction of the parasite.

In one embodiment, the 1,ω-bis(carboxy-phenoxy)(C₂-C₁₀)alkane is a 1,ω-bis(carboxy-phenoxy)(C₄-C₈)alkane. In another embodiment, the 1,ω-bis(carboxy-phenoxy)(C₂-C₁₀)alkane comprises 1,6-bis-(p-carboxyphenoxy)hexane (CPH) anhydrides. In yet another embodiment, the 1,ω-bis(carboxy)(C₂-C₁₀)alkane comprises sebacic anhydrides (SA). In one specific embodiment, the 1,ω-bis(carboxy)(C₂-C₁₀)alkane is sebacic anhydride (SA) and the 1,ω-bis(carboxyphenoxy)(C₂-C₁₀)alkane is 1,6-bis-(p-carboxyphenoxy)hexane (CPH).

In one embodiment, the ratio of 1,ω-bis(carboxy)(C₂-C₁₀)alkane units to 1,ω-bis(carboxyphenoxy)(C₂-C₁₀)alkane in the nanoparticle is about 90:10 to about 70:30. In another embodiment, the 1,ω-bis(carboxy)(C₂-C₁₀)alkane is sebacic anhydride (SA) and the 1,ω-bis(carboxyphenoxy)(C₂-C₁₀)alkane is 1,6-bis-(p-carboxyphenoxy)hexane (CPH) and the SA:CPH ratio is about 90:10 to about 70:30.

In one embodiment, the antiparasitic agent is ivermectin, abamectin, albendazole, amphotericin B, artimisinin, auranofin, chloroquine, diethylcarbamazine, eflornithine, emetine, halofantrine, mebendazole, mefloquine, metronidazole, miltofosine, moxidectin, piperazine, praziquantel, primaquine, proguanil, pyrantel pamoate, quinine, quinolones, rapamycin, spiramycin, suramin, thiabendazole, tinidazole, or a combination thereof. In various embodiments, antibiotic agent is amikacin, bacitracin, carbapenem, ceftiofur, chloramphenicols, ciprofloxacin, clindamycin, cycloserine, doxycycline, erythromycin, ethambutol, fluoroquinolones, gentamicin, isoniazid, rifampin, streptogramin, streptomycin, tetracycline, vancomycin, or a combination thereof.

In one specific embodiment, the antiparasitic agent is ivermectin and the antibiotic agent is doxycycline. In another specific embodiment, the antiparasitic agent comprises the combination of diethylcarbamazine and albendazole, and the antibiotic agent is doxycycline.

In one embodiment, the polyanhydride nanoparticle is capable of penetrating the surface (cuticle) of a parasitic worm to deliver the active agents to internal tissues of the parasite, while also killing endosymbiotic bacteria of the parasite.

In various embodiments, the polyanhydride nanoparticles kill parasites in less than three-fourths, less than one half, less than one quarter, or less than one tenth the time required for corresponding non-encapsulated active agents to kill the parasites at the same concentration of total active agents.

In one specific embodiment, the parasitic infection is lymphatic filariasis (Elephantiasis). In another specific embodiment, the parasitic infection is river blindness (Onchocerciasis). In yet another specific embodiment, the parasitic infection is caused by Brugia malayi or Brugia pahangi.

In another embodiment, the invention provides a method to deliver active agents to a mammal infected with parasites comprising: administering to a mammal infected by parasites an effective amount of a composition that includes polyanhydride nanoparticles and a combination of an antiparasitic agent and an antibiotic agent;

wherein the polyanhydride nanoparticles comprise copolymers of (a) 1,6-bis-(p-carboxyphenoxy)hexane (CPH) anhydride and sebacic anhydride (SA) in a ratio of about 10:90 to about 30:70; or (b) 1,8-bis(carboxyphenoxy)-3,6-dioxaoctane (CPTEG) anhydride and 1,6-bis-(p-carboxyphenoxy)hexane (CPH) anhydride in a ratio of about 10:90 to about 30:70; the nanoparticles are substantially spherical in shape, and have an average diameter of about 100 nm to about 900 nm;

the copolymers of the polyanhydride particles form a matrix around the antiparasitic agent and the antibiotic agent within the particles; and

the nanoparticles accumulate in the parasites in the mammal and degrade by surface erosion over a period of time to release the antiparasitic agent and the antibiotic agent, thereby delivering the agents to the parasites and killing or inhibiting the growth of the parasites. The antiparasitic agent can be, for example, ivermectin or the combination of diethylcarbamazine and albendazole, and the antibiotic agent can be, for example, doxycycline.

The invention further provides a polyanhydride nanoparticle and compositions thereof, wherein the nanoparticle comprises polyanhydride polymers in the form of a nanoparticle and a combination of two or more different active agents located in the interior of the nanoparticle, wherein the nanoparticle is substantially spherical in shape and has an average diameter of about 100 nm to about 900 nm; wherein the polyanhydride polymers comprise anhydride copolymers of 1,ω-bis(carboxy)(C₂-C₁₀)alkane units and 1,ω-bis(carboxyphenoxy)(C₂-C₁₀)alkane units; and wherein one of the active agents is an antiparasitic agent and a second active agent is an antibiotic agent. In one specific embodiment, the antiparasitic agent is ivermectin or the combination of diethylcarbamazine and albendazole, and the antibiotic agent is doxycycline.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention, however, one skilled in the art will understand that portions of the example or aspect may be used in combination with other examples or aspects of the invention.

FIG. 1. Rationale for using amphiphilic polyanhydride nanoparticles in therapies to treat filarial diseases.

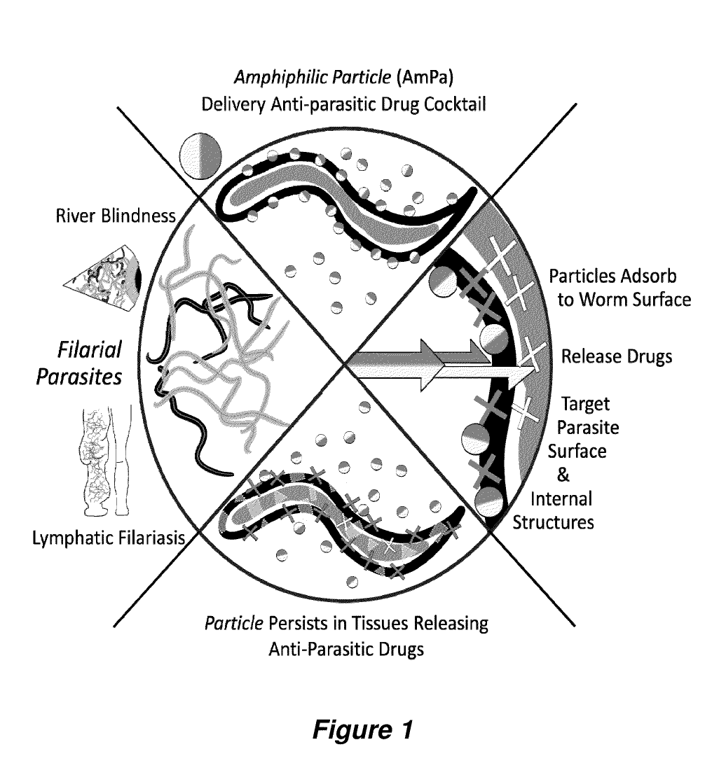

FIG. 2. PAN Delivery of Rhodamine Red inside B. malayi. Comparison of fluorescent rhodamine red distribution within worms treated with equivalent amounts of soluble vs. dye loaded into nanoparticles using laser scanning confocal microscopy (LSCM). Adult filarial worms were treated with equivalent amounts of either dissolved dye (for six days) or PAN-loaded dye (for 5 hours), fixed in 4% paraformaldehyde, and mounted onto glass slides. Nuclei of cells are counterstained with DAPI. No Rhodamine Red was observed inside B. malayi treated with soluble dye (upper right). Focused staining, consistent with high amounts of nanoparticles containing of dye, was found not only at the surface, but also under the cuticle and within deeper tissues in B. malayi treated with Rhodamine Red-encapsulated PANs (lower right). Below the lower right quadrant panel is a cross-sectional view of the lower right panel.

FIG. 3. (A) Cumulative drug release of actives from two representative polyanhydride nanoparticle, 20:80 CPH:SA (Nano A) and 20:80 CPTEG:CPH (Nano B). (B) Bright field image (left) and confocal fluorescence image (right) showing the presence of Rhodamine Red loaded PANs in the pharynx of B. malayi after 72 hours in 390 μM Nano A.

FIG. 4. Encapsulation of doxycycline and ivermectin into PANs (Nano A and Nano B), according to one embodiment.

FIG. 5. B. malayi Female High Dose (A) Average Time to Death (ATD) and (B) Motility.

FIG. 6. B. malayi (A) Female and (B) Male Low Dose ATD.

FIG. 7. B. malayi Male High Dose (A) ATD and (B) Motility.

FIG. 8. Reduction in microfilarial shedding.

FIG. 9. B. malayi Microfilaria Dose (A) ATD and (B) Motility.

FIG. 10. Average days to death for both B. malayi females (left half of graph) and males (right half of graph) after treatment with soluble IVM/Doxy directly compared to the formulations of PANs, according to some embodiments. N=5 per group with significance being p-value <0.05 for all treatment groups compared to the soluble drugs. Formulation A: 20:80 CPH:SA; Formulation B: 20:80 CPTEG:CPH; Formulation C: 50:50 CPTEG:CPH.

### DETAILED DESCRIPTION

The use of drug-loaded polyanhydride nanoparticles as an antiparasitic delivery vehicle improves penetration, and ultimately effectiveness, of the antiparasitic drugs for the treatment and killing of adult parasitic worms. We exploit this benefit of improved penetration to co-deliver in the nanoparticles an additional drug (e.g., an antibiotic such as doxycycline) that targets a symbiotic bacteria present naturally within the worm. Killing the symbiont results in death of the adult worm. Treating parasite infections with doxycycline alone is not practical due to the daily dosing regimen and cold storage that is required to maintain stability of the drug.

Polyanhydride nanoparticles can be used to co-encapsulate an antiparasitic agent (e.g., ivermectin) and an antimicrobial agent (e.g., doxycycline) to provide an effective therapeutic vector to kill and prevent reproduction of parasitic worms such as intestinal parasites. In effect, the antiparasitic-containing polyanhydride nanoparticles can attack several phases of the disease at once (killing adult worms and limiting reproduction of producing larvae that propagates the disease). The results described herein demonstrate a significant and dramatic improvement in killing parasitic worms and does so at smaller doses compared to currently used therapy, for example, oral or injectable doses of ivermectin and doxycycline for one month (followed by evaluating worm burden, and resuming treatment).

With the increased penetration of parasites afforded by polyanhydride nanoparticles, other infectious diseases caused by parasites can be treated by administration of the polyanhydride nanoparticles described herein. These diseases include both animal diseases (e.g., heartworm, hookworm, leishmania) and human diseases such as malaria, leishmoniasis, cryptosporidia, toxolasmosis, and Chagas disease. Endosymbionts including bacteria and algae/chloroplastscan also be killed by the therapeutic administration of the particles described herein. Plant nematodes can be treated and killed by contacting the infected plant and/or the soil surrounding the plant with an effective amount of the particles.

The inventors have discovered that nanoparticles of co-polymers of certain polyanhydrides (e.g., SA, CPH, and/or CPTEG) can penetrate the exterior of plant nematodes and intestinal parasites such as parasitic helminthes, worms, and flukes, Polyanhydride particles that are not co-polymers of polyanhydrides either do not penetrate the exterior of parasites to deliver effective amounts of active agent or they cannot be prepared and formulated as nanoparticles that encapsulate the actives. For example, a 50:50 ratio of CPH:SA polyanhydride polymers combined with actives in solution does not form nanoparticles but an amorphous mass. Thus, only a finite series of polyanhydride co-polymers have been found to be able to form suitable nanoparticles for the encapsulation of antiparasitic and antibiotic agents capable of penetrating the cuticle of parasites, as described herein. Suitable co-polymer particles that are effective for the methods described herein include CPH:SA (10:90-30:70), CPTEG:CPH (10:90-30:70), and CPTEG:CPH (40:60-60:40). A surfactant can be added to improve the processing and nanoparticle formation of the CPTEG:CPH (40:60-60:40) polymers, whereas the addition of a surfactant prevents the formation of suitable nanoparticles of CPH:SA (10:90-30:70) and CPTEG:CPH (10:90-30:70) compositions.

The invention thus provides a polyanhydride microparticle or nanoparticle that contains a plurality of active agents inside the particle; wherein the polyanhydride nanoparticle comprises anhydride copolymers of a 1,ω-bis(carboxy)(C₂-C₁₀)alkane and a 1,ω-bis(carboxyphenoxy)(C₂-C₁₀)alkane. The nanoparticle can be substantially spherical in shape and can have an average diameter of about 100 nm to about 900 nm. When the particle is a microparticle, the microparticle can be substantially spherical in shape and can have an average diameter of about 900 nm to about 5 μm.

The 1,ω-bis(carboxy)(C₂-C₁₀)alkane can be sebacic anhydride (SA), 1,8-bis(carboxyphenoxy)-3,6-dioxaoctane (CPTEG) anhydride, or a combination thereof. The 1,ω-bis(carboxyphenoxy)(C₂-C₁₀)alkane can be, for example, 1,6-bis-(p-carboxy-phenoxy)hexane (CPH), The polyanhydride nanoparticle is formed from anhydrides of these components for form copolymers. The ratio of 1,ω-bis(carboxy)(C₂-C₁₀)alkane to 1,ω-bis(4-carboxyphenoxy)(C₂-C₁₀)alkane in the nanoparticle can be about 90:10 to about 50:50 to about 10:90, or any ratio in between, such as 85:15, 80:20, 75:25, 70:30, 60:40, or 55:45, or the reverse of such ratios. Combinations of different ratio particles can be used (e.g., a formulations that includes a certain amount of 20:80 CPH:SA or CPTEG:CPH nanoparticles and also an additional amount of 50:50 CPTEG:CPH nanoparticles; in mass ratios of about 10-90% of the 20:80 nanoparticles to about 90-10% 50:50 nanoparticles).

In certain specific embodiments, the 1,ω-bis(carboxy)(C₂-C₁₀)alkane is sebacic anhydride (SA) and the 1,ω-bis(carboxyphenoxy)(C₂-C₁₀)alkane is 1,6-bis-(p-carboxyphenoxy)hexane (CPH). The 1,ω-bis(carboxy)(C₂-C₁₀)alkane can also be 1,8-bis(carboxyphenoxy)-3,6-dioxaoctane (CPTEG) and the 1,ω-bis(carboxyphenoxy)(C₂-C₁₀)alkane can be 1,6-bis-(p-carboxyphenoxy)hexane

Thus, the invention provides a method to treat a parasitic infection in an animal or plant comprising administering to an animal or plant in need of such treatment an effective antiparasitic amount of a composition comprising polyanhydride nanoparticles or microparticles. These particles can comprise a combination of active agents such as antiparasitic agents and antibiotic agents. The nanoparticles can accumulate in the parasite, and can degrade by surface erosion over a period of time to release the active agents so as to contact and kill the parasite or inhibit the growth of the parasite causing the infection, thereby treating the parasitic infection. The nanoparticles can also inhibit the production of and/or kill offspring (e.g., microfilaria and larvae).

The polyanhydride nanoparticles can include a combination of two or more different active agents located in the interior of the nanoparticle. The nanoparticles can be substantially spherical in shape and a set of particles can have an average diameter of about 100 nm to about 900 nm, or about 200 nm to about 500 nm. The particles can also be prepared as microparticles having average diameters of about 1 μm to about 10 μm, or about 1 μm to about 5 μm. These larger particles can be used to treat plant seeds (e.g., soybeans) or the soil surrounding plants, for example, to prevent or inhibit infection or to treat a plant nematode infection.

The polyanhydride nanoparticles can comprise anhydride copolymers of 1,ω-bis(carboxy)(C₂-C₁₀)alkane units and 1,ω-bis(carboxyphenoxy)(C₂-C₁₀)alkane units. One of the active agents can be an antiparasitic agent and a second active agent can be an antibiotic agent.

In some embodiments, the 1,ω-bis(carboxy-phenoxy)(C₂-C₁₀)alkane is a 1,ω-bis(carboxy-phenoxy)(C₄-C₈)alkane.

In some embodiments, the 1,ω-bis(carboxy-phenoxy)(C₂-C₁₀)alkane comprises 1,6-bis-(p-carboxyphenoxy)hexane (CPH) anhydrides.

In some embodiments, the 1,ω-bis(carboxy)(C₂-C₁₀)alkane comprises sebacic anhydrides (SA).

In some embodiments, the 1,ω-bis(carboxy)(C₂-C₁₀)alkane is sebacic anhydride (SA) and the 1,ω-bis(carboxyphenoxy)(C₂-C₁₀)alkane is 1,6-bis-(p-carboxyphenoxy)hexane (CPH).

In some embodiments, the ratio of 1,ω-bis(carboxy)(C₂-C₁₀)alkane units to 1,ω-bis(carboxyphenoxy)(C₂-C₁₀)alkane in the nanoparticle is about 90:10 to about 50:50.

In some embodiments, the 1,ω-bis(carboxy)(C₂-C₁₀)alkane is sebacic anhydride (SA) and the 1,ω-bis(carboxyphenoxy)(C₂-C₁₀)alkane is 1,6-bis-(p-carboxyphenoxy)hexane (CPH).

In various embodiments, the ratio of polyanhydrides is about 90:10 to about 70:30, or about 80:20. In other embodiments, the ratio of polyanhydrides is about 60:40 to about 40:60, or about 50:50. In one specific embodiment, the SA:CPH ratio is about 90:10 to about 70:30, or about 80:20. In another specific embodiment, the CPTEG:CPH ratio is about 10:90 to about 30:70, or about 20:80. In yet another specific embodiment, the CPTEG:CPH ratio is about 60:40 to about 40:60, or about 50:50.

The invention thus provides a method to treat infections of parasitic helminthes, worms, or flukes. In some embodiments, the parasite is an intestinal parasites or a plant nematode. For treating plant nematodes, the soil surrounding an infected plant, or a plant that could be infected, can be treated with a composition comprising the active agent-loaded particles described herein.

In some embodiments, the antiparasitic agent is ivermectin, diethylcarbamazine, albendazole, moxidectin, or a combination thereof.

In some embodiments, the antimicrobial agent (e.g., antibiotic agent) comprises amikacin, bacillomycin, cephalexin, cephalosporin, ciprofloxacin, doxycycline, erythromycin, ethambutol, gentamicin, a heavy metal such as ions of Ag, As, Cd, Co, Cu, Hg, Mn, Ni, or Zn, isoniazid, penicillin, rifampin (rifampicin), spectinomycin, streptomycin, sulfa, tetracycline, trimethoprim-sulfamethoxazole, vancomycin, or a combination thereof.

In one specific embodiment, the antiparasitic agent is ivermectin and the antimicrobial agent is doxycycline.

In some embodiments, the polyanhydride nanoparticle is capable of penetrating the surface (cuticle) and interior tissues of a parasitic worm. The nanoparticles can also be ingested by parasitic species, thereby enhancing the treatment by proving the actives by a second mechanism.

The polyanhydride nanoparticles can encapsulate effective antiparasitic and antibacterial amounts of active agents sufficient to treat a parasitic infection. The treatments can include killing parasites, inhibiting reproduction, and killing offspring such as microfilaria and larvae. A composition of the nanoparticles can be administered to provide various concentrations of the actives to the parasites. For example, 100 μg of each active agent can be provided in 2 mg of nanoparticles (5% loading of each active agent) to provide a 195 μM formulation. Higher doses can also be administered to animals. For example, infected animals have been treated with 10 mg of nanoparticles in a 0.5 mL aqueous formulation, where the nanoparticles contained 1 mg of each active agent (10% loading of each active). Suitable formulations can include about 0.1 mg/mL to about 2 mg/mL of active agent. Suitable effective dosages for animals can be about 0.05 mg of nanoparticles/kg to about 500 mg of nanoparticles/kg, where the nanoparticles contain about 1-10 wt. % of each active agent.

The invention also provides methods to kill parasites or inhibit the growth of parasites comprising: contacting the host of a parasite with a composition comprising an effective amount of polyanhydride nanoparticles as described herein. The nanoparticles can degrade by surface erosion in the presence of the parasite over a period of time to release active agents from the interior of the nanoparticles, thereby killing the parasite or inhibiting the growth of the parasite.

In some embodiments, the parasitic infection is lymphatic filariasis (Elephantiasis).

In some embodiments, the parasitic infection is river blindness (Onchocerciasis).

In some embodiments, the parasitic infection is caused by Brugia malayi or Brugia pahangi.

The particles can be particularly effective for the killing of female parasites such as female Brugia worms. In some embodiments, a composition of the particles described herein can be as effective as a 100× concentration of the corresponding soluble drug or drugs for killing male parasitic worms over the same time period (e.g., three days). In other embodiments, a composition of the particles described herein can be as effective as a 100× concentration or a 1000× concentration of the corresponding soluble drug or drugs for killing female parasitic worms over the same time period (e.g., three days).

The invention yet further provides methods to deliver active agents to a mammal infected with parasites comprising: contacting the mammal infected by parasites with an effective amount of a composition that includes polyanhydride nanoparticles and a combination of an antiparasitic agent and an antimicrobial agent. The polyanhydride nanoparticles can comprise copolymers of (a) sebacic anhydride (SA), and (b) 1,6-bis-(p-carboxyphenoxy)hexane (CPH) anhydride in a ratio of about 80:20 to about 50:50. The nanoparticles can be substantially spherical in shape, and can have an average diameter of about 100 nm to about 900 nm. The copolymers of the polyanhydride particles can form a matrix around the active agents within the particles; and the nanoparticles can accumulate in the parasites in the mammal and degrade by surface erosion over a period of time to release the antiparasitic agents and the antimicrobial agents, thereby delivering the agents to the parasites and killing or inhibiting the growth of the parasites. In various embodiments, the nanoparticles penetrate the outer membrane (cuticle) of the parasite, for example, a parasitic worm.

The antiparasitic agent can be ivermectin or another antiparasitic agent described herein. The antimicrobial agent can be doxycycline or another antimicrobial agent described herein. The nanoparticle can further include a second antimicrobial agent and/or a second antiparasitic agent.

Many parasites such as helminths possess endoparasitic bacterium (Wolbachia) Killing the endoparasitic bacteria results in death of the helminths. However, current methods of killing the endoparasitic bacterium of helminths takes approximately one year of regular administration of antibiotics. Patient compliance for such long treatment periods is problematic. Accordingly, new therapies are urgently needed. The nanoparticle formulations described herein solve these problems. The dual-drug loaded nanoparticle formulations possess a mechanism of action by which both antiparasitic actives and antibiotic actives can be administered to a subject to achieve significantly enhanced killing of parasites and the endoparasitic bacteria compared to soluble drugs.

### Polyanhydride Nanoparticle Delivery Platform Enables Enhanced Killing of Parasitic Helminthes, Worms, and Flukes.

Lymphatic filariasis and related infections represent a significant global burden, endemic in over 80 countries worldwide, particularly India and Sub-Saharan Africa, and infecting up to 120 million individuals. These parasitic infections can cause diseases such as lymphedema, hydrocele, and elephantiasis. By using a standard anti-parasitic drug, ivermectin, which acts as a microfilaricide, and the antimicrobial, doxycycline, to target its symbiotic bacteria Wuchereria bancrofti as well as to act as a macrofilaricide, polyanhydride nanoparticles can be used as an effective drug delivery vector, reducing the amount of drug necessary for macrofilarial death by up to 100-fold.

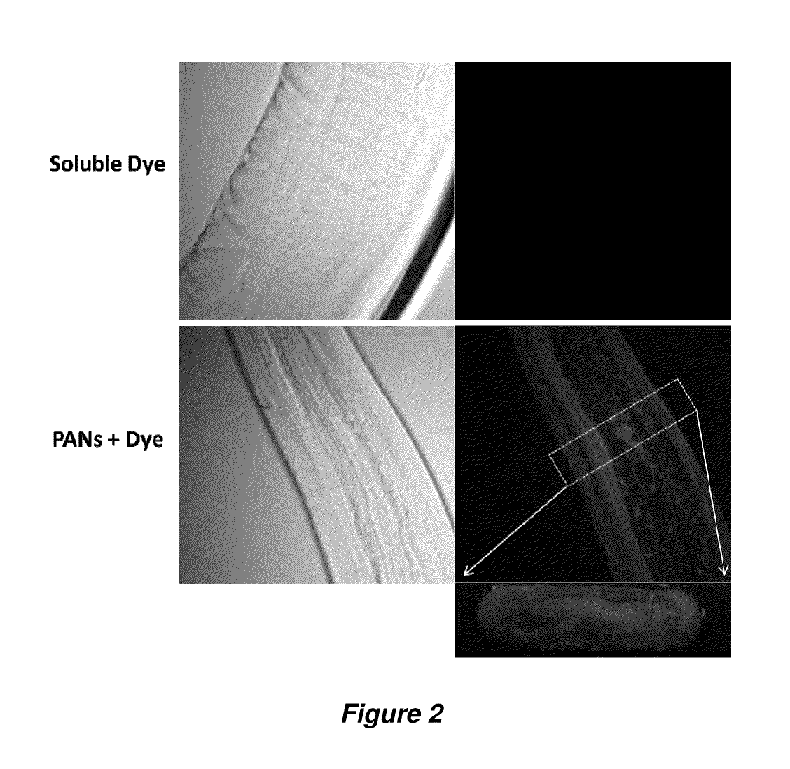

Encapsulation of the anti-LF drug ivermectin, and the antimicrobial doxycycline, into polyanhydride nanoparticles increased the efficacy of treatments. The polyanhydride nanoparticle drug delivery platform decreased the average time to death of B. malayi females and facilitated a more rapid decrease in the motility of treated worms, with similar trends seen at nanomolar concentrations and in the B. malayi males. Additionally, at low doses of drug, encapsulation into the polyanhydride nanoparticles described herein reduced the amount of microfilaria shed over the course of the experiment. Finally, confocal microscopy images begin to provide an explanation of the interaction and efficacy profile of polyanhydride nanoparticle drug delivery to parasitic worms. Tracking a fluorescent dye co-loaded into the particles demonstrates a greater increased influx of payload into the worm compared to attempted delivery of solubilized drug and dye, as shown in FIG. 2.

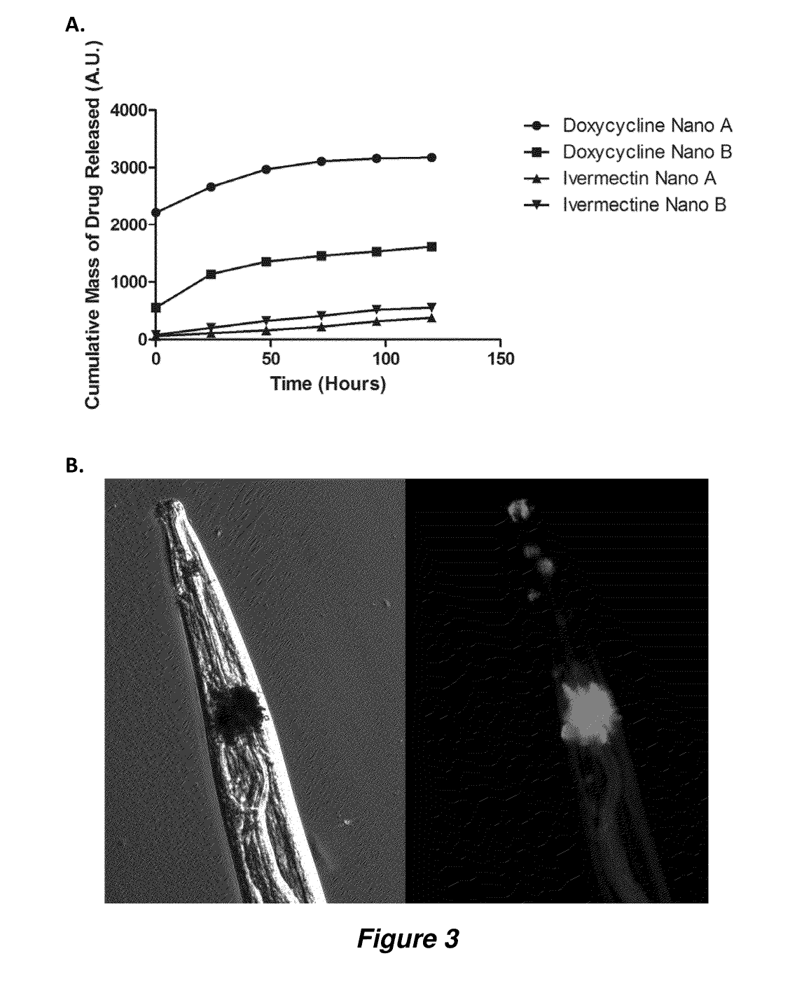

The unique chemistry of polyanhydride nanoparticles can be applied to address many of the challenges associated with mass drug administration against parasitic infections such as lymphatic filariasis. The surface erosion profile of the polyanhydride nanoparticles provides sustained release of drug over an extended time profile. The sustained release of doxycycline and ivermectin from the chemistries of two representative polyanhydride nanoparticle, 20:80 CPH:SA (Nano A) and 20:80 CPTEG:CPH (Nano B), is shown in FIG. 3. A larger initial burst of the doxycycline is observed from the 20:80 CPH:SA, consistent with our previous work. Additionally, a distinct release profile is observed from the release of ivermectin from both polyanhydride chemistries, characterized by a very low burst, followed by a zero-order release over the course of the treatment.

In addition to providing sustained release of drug, with implications for dose sparing and increased patient compliance, the polyanhydride nanoparticle drug delivery vector demonstrated unique interactions with parasitic worms such as the B. malayi worms. One of the benefits of nanoparticle delivery is the ability to create a high drug concentration microenvironment, in contrast to soluble drugs, which diffuse in aqueous solutions and environments. To achieve the benefit of an increased drug concentration, the nanoparticle must interact with a target cell, or, in this case, the microfilaria or adult worm. Confocal microscopy indicates that the nanoparticles are interacting with the worms in a way that the soluble drugs and dye are unable to do. The nanoparticles allow facilitated active agent diffusion into the worm, by embedding in membrane barriers and then carrying the drug payload through the membrane barriers. Facilitated diffusion is a recognized transport mechanism of drugs and other substances normally done by embedded proteins, which is achieved by the biodegradable nanoparticles described herein.

Filarial diseases represent a significant social and economic burden in areas that are endemic with filarial endoparasite B. malayi, and its symbiotic bacteria Wuchereria bancrofti. The invention provides for the use of a polyanhydride nanoparticle-based drug delivery platform for the co-delivery of antiparasitic drugs to reduce the macro- and microfilarial burden and antimicrobial drugs to eliminate the symbiotic bacteria, Wolbachia. Examples of the antiparasitic that can be co-delivered include ivermectin, moxidectin, mebendazole, pyrantel pamoate, thiabendazole, albendazole, praziquantel, amphotericin B, miltofosine, eflornithine, tinidazole, metronidazole, chloroquine, primaquine, mefloquine, proguanil, emetine, rapamycin, artimisinin, and other antiparasitic active agents described herein. Examples of antibiotics that can be co-delivered include doxycycline, rifampin, amakacin, gentamicin, ciprofloxacin, ceftiofur, erythromycins, tetracyclines, chloramphenicols, fluoroquinolones, and other antibiotics described herein. Combinations of one or two antiparasitic can be included, and combinations of one or two antibiotics can be included in the same particle or in a composition of particles, thereby providing a variety of two, three, and four-drug particles or particle compositions.

The co-delivery of antiparasitic drugs and antibiotics in polyanhydride nanoparticles (PANs) effectively killed adult B. malayi filarial worms with up to a 100-fold reduction in the amount of drug used. Further, the time to death of the macrofilaria was significantly reduced when the anti-filarial drug cocktail was delivered by PANs. Confocal microscopy shows that the mechanism behind this enhanced killing of the macrofilaria includes the ability of the PANs to positively interact and adhere with the cuticle, penetrate the outer membrane, delivery of the patristic drugs to vital areas within B. malayi worm, and effectively deliver drugs at high enough microenvironment concentrations to cause death. Additional observations indicated that parasites ingest the nanoparticles, resulting in fast transport of the nanoparticles internally, resulting in faster host parasite death. These findings may have significant consequences for the reducing the amount of drug and the length of treatment required for filarial infections.

### DEFINITIONS

As used herein, certain terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, “an example embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect such aspect, feature, structure, moiety, or characteristic in connection with other embodiments, whether or not explicitly described.

The term “and/or” means any one of the items, any combination of the items, or all of the items with which this term is associated.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer. Unless indicated otherwise herein, the term “about” is intended to include values, e.g., weight percentages, proximate to the recited range that are equivalent in terms of the functionality of the individual ingredient, the composition, or the embodiment.

As will be understood by one skilled in the art, for any and all purposes, particularly in terms of providing a written description, all ranges disclosed herein also encompass any and all possible subranges and combinations of subranges thereof as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, tenths, etc. As a non limiting example, each range discussed herein can be readily broken down into a lower third, middle third and upper third, etc. As will also be understood by one skilled in the art, all language such as “up to,” “at least,” “greater than,” “less than,” “more than,” “or more” and the like include the number recited and refer to ranges which can be subsequently broken down into subranges as discussed above. In the same manner, all ratios disclosed herein also include all sub-ratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Additionally, for all purposes, the present invention encompasses not only the main group, but also the main group absent one or more of the group members. The present invention also envisages the explicit exclusion of one or more of any of the group members in the claimed invention.

As will be understood by the skilled artisan, all numbers, including those expressing quantities of ingredients, properties such as molecular weight, reaction conditions, and so forth, are approximations and understood as being modified in all instances by the term “about.” These values can vary depending upon the desired properties sought to be obtained by those skilled in the art utilizing the present teachings of the present invention. It is also understood that such values inherently contain variability necessarily resulting from the standard deviations found in their respective testing measurements.

The phrase “one or more” is readily understood by one of skill in the art, particularly when read in context of its usage. For example, one or more substituents on a phenyl ring refers to one to five, or one to up to four, for example if the phenyl ring is disubstituted.

The terms “polyanhydride particle” and “polyanhydride nanosphere” both refer to microparticles and nanoparticles made of polyanhydride polymers as described herein. The polyanhydride polymers of the particles are typically copolymers, such as random mixes of anhydride oligomers (condensed prepolymers). The polyanhydride particle can be abbreviated as “PA particle”, which can be a microparticle or a nanoparticle. The nanoparticles can be referred to as polyanhydride nanosphere (PANS).

The term “polymer” refers to a molecule of one or more repeating monomeric residue units covalently bonded together by one or more repeating chemical functional groups. The term includes all polymeric forms such as linear, branched, star, random, block, graft and the like. It includes homopolymers formed from a single monomer, copolymers formed from two or more monomers, terpolymers formed from three or more polymers and other polymers formed from more than three monomers. Differing forms of a polymer may also have more than one repeating, covalently bonded functional group.

The term “polyanhydride” refers to a polymer that is derived from the condensation of carboxylic acids or carboxylic acid derivatives such that repeating units of the resulting polymer are linked by anhydride (—C(═O)—O—C(═O)—) groups. Polyanhydrides can be prepared by condensing diacids or by condensing anhydride prepolymers.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo. For example, a parasite can be killed or inhibited from growing or reproducing when contacted with an antiparasitic agent.

An “effective amount” refers to an amount effective to treat a disease, disorder, and/or condition, or to bring about a recited effect. For example, an amount effective can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes both medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The particles described herein can encapsulate a variety of types of cargo by incorporating the cargo molecules into the polyanhydride matrix of the particles. The particles can readily incorporate two or more different types of active agents. Co-agents and/or additives such as dyes and radioactive nuclei may be included in the particles for diagnostic purposes. Other additives can include compounds such as bacillomycin, which can enhance the activity of the active agent. Additionally, bacillomycin can be added to the particle formulation such that it is included inside the particles with a primary active agent, and outside the particle, in the pharmaceutical solution or linked to the particle covalently by a linker such as PEG.

Accordingly, the particles can be loaded with a variety of different active agents. The term “active agent” (and its equivalents “agent,” “drug,” “bioactive agent,” “medicament” and “pharmaceutical”) is intended to have the broadest meaning and includes at least one of any therapeutic, prophylactic, pharmacological or physiological active substance, cosmetic and personal care preparations, and mixtures thereof, which is delivered to an animal or plant to produce a desired, usually beneficial, effect. More specifically, any active agent that is capable of producing a pharmacological response, localized or systemic, irrespective of whether therapeutic, diagnostic, cosmetic or prophylactic in nature, is within the contemplation of the invention. Bioactive agents such as antiparasitic agents, antibacterial agents, pesticides, insect repellents, sun screens, cosmetic agents, and the like may be encapsulated by the particles.

It should be noted that the drugs and/or bioactive agents may be used singularly or as a mixture of two or more such agents, and in amounts sufficient to prevent, cure, diagnose or treat a disease or other condition, as the case may be. The drugs and mixtures thereof can be present in the composition in different forms, depending on which form yields the optimum delivery characteristics. Thus, in the case of drugs, the drug can be in its free base or acid form, or in the form of salts, esters, amides, prodrugs, enantiomers or mixtures thereof, or any other pharmacologically acceptable derivatives, or as components of molecular complexes.

In various embodiments, the active agent can be, for example, an antiparasitic agent, antimicrobial agent, or a combination thereof. The term “antiparasitic agent” refers to bioactive molecules that kill or inhibit the growth or replication of nematodes, cestodes, trematodes, infectious protozoa, and amoebas, or that treat conditions and diseases caused by such parasites. The term “antimicrobial agent” refers to bioactive molecules that kill or inhibit the growth or replication of bacteria, fungi, algae, or other pathogenic organisms, such as tuberculosis. Examples of drugs and antimicrobial agents that can be encapsulated in the particles described herein include amikacin, doxycycline, and the generic and specific agents listed at paragraphs [0057] to [0342] of U.S. Patent Publication No. 2006/0078604 (Kanios et al.), which paragraphs are incorporated herein by reference.

Additional examples of antimicrobial agents include sulfonamides, beta-lactams including penicillin, cephalosporin, and carbepenems, aminoglycosides, quinolones, and oxazolidinones, and metals such as copper, iron, aluminum, zinc, gold, compound and ions thereof, and various combinations thereof. Other agents that can be included in the polyanhydride particles include lipopolysaccharides (LPS), polyguanidines (CPG), bacterial lysates, such as material from a slurry of heat killed Brucella, e.g., to form a vaccine, and multi kDa proteins, such as defensins (cysteine-rich cationic proteins of about 18-45 amino acids).

Specific values listed herein for radicals, substituents, ranges, and other described values are for illustration only; they do not exclude other recited values or other values within defined ranges for components, in various embodiments. In other embodiments, any recited value or range may be excluded from the scope of an embodiment.

### Polyanhydride Prepolymers, Polymers, and Synthesis Thereof.

The polyanhydrides used to prepare the particles of the invention can be prepared as described herein or by methods known to those of skill in the art. A number of examples of methods for the preparation of polyanhydrides are provided below. A wide range of suitable diacids can be employed to prepare polyanhydrides. The diacid can be a diacid-substituted straight or branched chain alkane that is optionally interrupted by about one to about five -Ph-, —O—, —CH═CH—, and/or —N(R)— groups wherein R is H, phenyl, benzyl, or (C₁-C₆)alkyl. In one embodiment, the alkane of the diacid can be C₂-C₁₂(alkyl). In another embodiment, the alkane can be C₄-C₈(alkyl). Additionally, the alkane group of the diacid can be optionally interrupted by about 1 to about 12 —OCH₂CH₂O— groups, for example, a poly(ethylene glycol) segment. The alkane group can also be optionally substituted with one, two, or three (C₁-C₆)alkyl, (C₁-C₆)alkenyl, trifluoromethyl, trifluoromethoxy, or oxo groups; or combinations thereof.

In one embodiment, a prepolymer can be prepared as illustrated in Scheme 1.

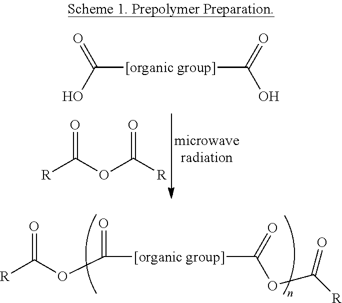

where “organic group” is any organic group that can link two carboxylic acid moieties, R is alkyl or aryl, and n is 1 to about 12. Examples of suitable organic groups include, but are not limited to, C₂-C₁₂(alkyl) groups, -PhO—C₂—C₁₂(alkyl)-OPh- groups, and PEG groups having 1 to about 12 PEG units, such as a 3,6-dioxaoctane group. A molar excess of the carboxylic anhydride can be employed. About 2 to about 30 molar equivalents of the carboxylic anhydride can be used. Alternatively, about 5 to about 20 molar equivalents of the carboxylic anhydride can be used. In one embodiment, 6 molar equivalents of the carboxylic anhydride are employed. In another embodiment, 18 molar equivalents of the carboxylic anhydride are employed. The carboxylic anhydride can be, for example, acetic anhydride, trifluoroacetic anhydride, benzoic anhydride, combinations thereof, and/or derivatives thereof.

A prepolymer can also be prepared as illustrated in Scheme 2.

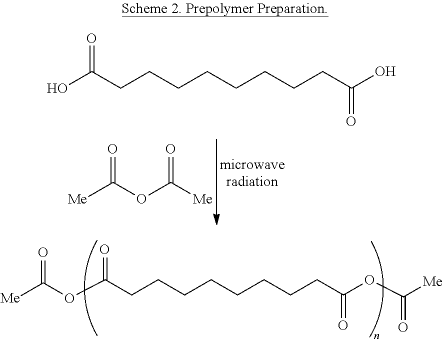

wherein n is 1 to about 12. Other carboxylic anhydrides can be used to form the end groups of the prepolymer, such as, but not limited to, benzoic anhydride. The central aliphatic group can optionally be substituted or interrupted as described herein.

The diacid can also be a 1,ω-bis(carboxy)alkane. As would be recognized by one skilled in the art, alternative nomenclature for a 1,ω-bis(carboxy)alkane is a 1,ω-alkanedioic acid that has two additional carbons in the alkane moiety compared to the corresponding bis(carboxy)alkane.

A prepolymer can also be prepared as illustrated in Scheme 3.

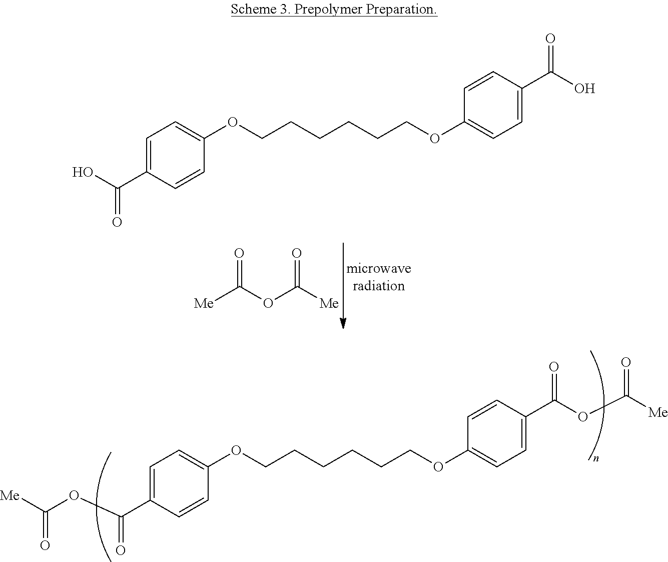

wherein n is 1 to about 12. Carboxylic anhydrides other than acetic anhydride can be used to form the end groups of the prepolymer. The central aliphatic group, the aryl groups, or both, can optionally be substituted, in any combination. The central aliphatic group can also be interrupted by oxygen, for examples, as with a poly(ethylene glycol) chain.

Accordingly, the diacid can be two aryl groups that are each substituted with a carboxy group wherein the aryl groups are linked by a straight or branched chain alkane that is optionally interrupted by about one to about five -Ph-, —O—, —CH═CH—, and/or —N(R)— groups wherein R is H, phenyl, benzyl, or (C₁-C₆)alkyl. In some embodiments, one or both of the aryl groups can be omitted and the carboxy groups are linked by the alkyl chain. In one embodiment, the alkane can be C₂-C₁₂(alkyl). In another embodiment, the alkane can be C₄-C₈(alkyl). In another embodiment, the alkane can be one or more PEG groups. Additionally, the alkane group linking the carboxylic acid-substituted aryl groups can be optionally interrupted by 1 to about 12 —OCH₂CH₂O— groups, for example, a poly(ethylene glycol) segment. The alkane group linking the carboxylic acid-substituted aryl groups can also be optionally substituted with one, two, or three (C₁-C₆)alkyl, (C₁-C₆)alkenyl, trifluoromethyl, trifluoromethoxy, or oxo groups; or combinations thereof.

The diacid can be a 1,ω-bis(4-carboxyphenoxy)alkane. In one embodiment, the alkane is a (C₂-C₁₀)alkane. In another embodiment, the alkane can be a C₄-C₈(alkyl). In certain specific embodiments, alkane can be ethyl, propyl, butyl, pentyl, hexyl, heptyl, octyl, and branched isomers thereof. In one embodiment, the diacid is a 1,6-bis(4-carboxyphenoxy)hexane. In another embodiment, the diacid is a 1,6-bis(carboxy)octane. In another embodiment, the diacid can be a 1,8-bis(carboxyphenoxy)-3,6-dioxaoctane. Mixtures of any of these diacids can be used in conjunction with the microwave facilitated methods described herein.

Polyanhydrides.

Polyanhydrides can be prepared by condensation methods known in the art or by irradiating a prepolymer with a sufficient amount of microwave irradiation to polymerize the prepolymer. A sufficient amount of microwave radiation can typically be generated by a conventional microwave oven set to 1100 Watts for about 1 to about 30 minutes. More often, a sufficient amount of microwave radiation can be generated in about 1 to about 20 minutes. The resulting polyanhydride can be a homopolymer or a copolymer, depending on the nature of the prepolymer composition used in the reaction.

A polyanhydride can also be prepared by forming a prepolymer in situ from diacids. The diacids can be converted into prepolymers by irradiating diacids in the presence of a carboxylic anhydride. The prepolymer can be prepared by, for example, by irradiating a mixture of (a) a carboxylic anhydride and (b) an aromatic dicarboxylic acid, an aliphatic dicarboxylic acid, or a mixture thereof, with an amount of microwave radiation effective to form the prepolymer. One suitable carboxylic anhydride is acetic anhydride. Other suitable carboxylic anhydrides include, for example, trifluoroacetic anhydride and benzoic anhydride.

The terminal groups of polyanhydrides prepared according to the methods described herein will typically have terminal acyl groups. It is possible for some hydrolysis of the polyanhydrides to occur during the reaction or during the isolation of the polyanhydride. Thus, some terminal groups of such polyanhydrides can be carboxylic acid groups. Accordingly, the methods of the invention include the preparation of polyanhydrides that terminate in acyl groups, carboxylic acid groups, or combinations thereof.

The polyanhydride can be prepared, for example, as illustrated in Scheme 4.

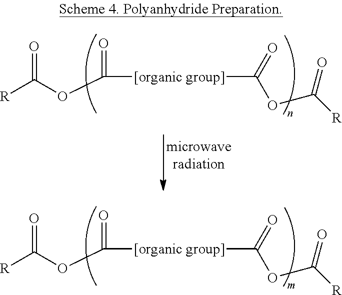

where “organic group” is any organic group that links two carboxylic acid moieties, R is alkyl or aryl, n is 1 to about 12, and m is about 5 to about 200.

The polyanhydride can also be prepared as illustrated in Scheme 5.

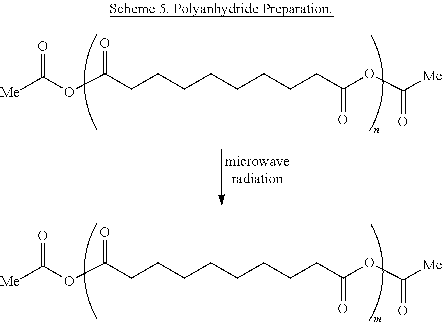

where n is 1 to about 12 and m is about 5 to about 200. In other embodiments, m can be about 10 to about 100, or about 10 to about 50. As would be understood by one skilled in the art, the value of m will typically be larger than the value of n. End groups other than acetate can be used and the central aliphatic group can be optionally substituted or optionally interrupted (e.g., as for PEG groups), or both, as described herein.

The polyanhydride can also be prepared as illustrated in Scheme 6.

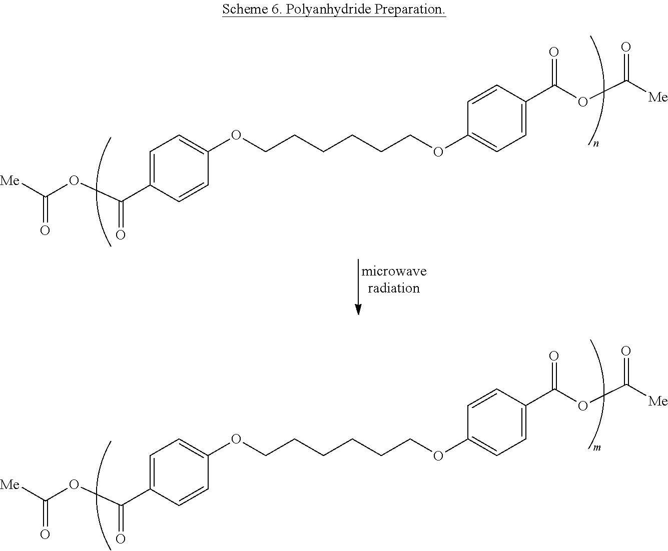

wherein n is 1 to about 12 and m is about 5 to about 100. In other embodiments, m can be about 10 to about 50, or about 15 to about 35. End groups other than acetate can be used and the central aliphatic group, the aryl groups, or both, can optionally be substituted, in any combination. The central aliphatic group can also be optionally interrupted by oxygen, for examples, as with a poly(ethylene glycol) chain.

### Polyanhydride Polymers for Preparation of Microparticles and Nanoparticles.

A method for preparing the polyanhydride microparticles or nanoparticles includes irradiating one or more diacids, wherein the one or more diacids include an aromatic dicarboxylic acid, an aliphatic dicarboxylic acid, or a mixture thereof, with microwave radiation in the presence of a carboxylic anhydride so as to acylate one or more diacids to yield at least one prepolymer; and irradiating the prepolymer with microwave radiation so as to polymerize said prepolymer to yield the polyanhydride, as a homopolymer or a copolymer.

The prepolymers can be made up of dicarboxylic acids (“diacids”) that are acylated at both acid moieties. A prepolymer can be a single acylated diacid unit (monomer), or it can have up to about 12 condensed diacid units. A mixture of different diacids can be employed. The mixture of diacids can yield a random copolymer. The one or more diacids can include a diacid-substituted C₂-C₁₂ straight or branched chain alkane that is optionally interrupted by about 1 to about 5 -Ph-, —O—, —CH═CH—, and/or —N(R)— groups wherein R is H, phenyl, benzyl, or (C₁-C₆)alkyl. The one or more diacids can also be optionally interrupted by about 1 to about 12 —OCH₂CH₂O— groups. The one or more diacids can also be optionally substituted with 1, 2, or 3 trifluoromethyl, trifluoromethoxy, (C₁-C₆)alkyl, (C₁-C₆)alkenyl, or oxo groups, or combinations thereof.

The at least one diacid can be a 1,ω-bis(carboxy)alkane. The at least one diacid can also be a 1,ω-bis(4-carboxyphenoxy)alkane. The alkane can be, for example, a (C₃-C₈)alkane. Specific examples of the alkane include hexane and octane. The diacid can be 1,6-bis(4-carboxyphenoxy)hexane. Alternatively, the diacid can be 1,6-bis(carboxy)octane (sebacic acid). The at least one prepolymer can also include a bis(carboxylic acid acetyl ester), or an anhydride oligomer thereof. The at least one prepolymer can also include a 1,ω-(4-acetoxycarbonylphenoxyl)alkane, or an anhydride oligomer thereof, or a 1,8-bis(carboxyphenoxy)-3,6-dioxaoctane, or an anhydride oligomer thereof.

The carboxylic anhydride can be a bis-alkyl carboxylic anhydride, a bis-aryl carboxylic anhydride, an alkyl-aryl carboxylic anhydride, or a mixture thereof. The carboxylic anhydride can be, for example, acetic anhydride, trifluoroacetic anhydride, or benzoic anhydride. A molar excess of the carboxylic anhydride can be employed. Excess carboxylic anhydride can be removed after the prepolymer has formed.

In various embodiments, the polymers of the microparticles and/or nanoparticles described herein can be poly-sebacic anhydrides (SA), poly-1,6-bis-(p-carboxyphenoxy)hexane (CPH) anhydrides, or poly-1,8-bis(carboxyphenoxy)-3,6-dioxaoctane (CPTEG) anhydrides. In other embodiments, the polymers of the microparticles and/or nanoparticles described herein can be copolymers of sebacic anhydride (SA) and 1,6-bis-(p-carboxyphenoxy)hexane (CPH) anhydride, or copolymers of 1,8-bis(carboxyphenoxy)-3,6-dioxaoctane (CPTEG) anhydride and 1,6-bis-(p-carboxyphenoxy)hexane (CPH) anhydride. The ratio of SA to CPH, or CPTEG to CPH, can be any integer from about 1:19 to about 19:1. An example of a structure of a SA:CPA copolymer is:

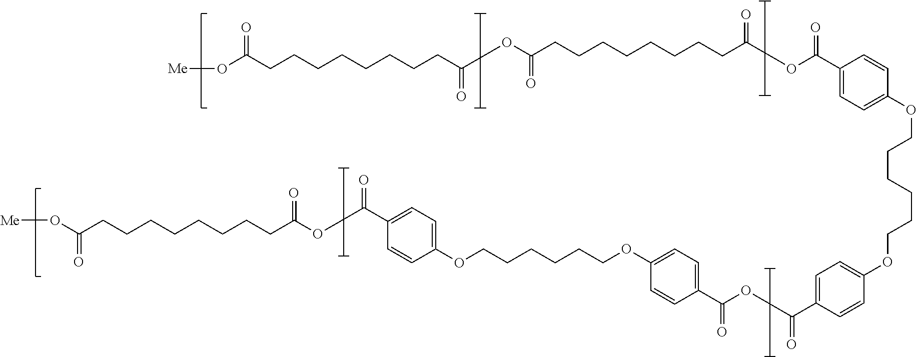

where each block (designated by a single or double bracket) includes a number of repeating units sufficient to provide a polymer with an Mₙ of about 5,000 to about 50,000 g/mol, such as about 10,000 to about 25,000 g/mol, or about 15,000 to about 20,000 g/mol. The anhydride copolymer can be a block copolymer or a random copolymer, or a combination thereof. CPTEG:CPH copolymers can also be prepared to form polymers where each block can include a number of repeating units sufficient to provide a polymer with an Mₙ of about 5,000 to about 50,000 g/mol, such as about 10,000 to about 25,000 g/mol, or about 15,000 to about 20,000 g/mol.

The PA particles, or polyanhydride nanoparticles (PANs), described herein can be loaded with an effective amount of one or more active agents. The PANs have been loaded with doxycycline, numerous model agents, and various combinations thereof. This successful loading indicates that any active agent, including both hydrophilic and hydrophobic agents, and be effectively encapsulated without agent deterioration. For example, PANs that have encapsulated doxycycline with either 1.5% or 3% loading kill laboratory and field strains of Brucella canis and laboratory strains of Escherichia coli by agar disk diffusion assays. Once administered to infected cells, the PANs can localize in intracellular compartments that contain an intracellular pathogen. This localization can occur without killing the host cell that internalized the PANs.

The particles described herein can be used in combination with two or more actives to kill parasites and treat parasitic infections. The polymers and particles described by U.S. Pat. Nos. 8,449,916, 8,173,104, and 7,858,093, each incorporated herein by reference, can also be used with the methods described herein, in various embodiments. Parasites that can be killed with the polyanhydride particles include parasitic helminthes, worms, and flukes, including plant nematodes and various intestinal parasites. Examples of parasites that can be killed and conditions that can be treated include Soil Transmitted Helminths (e.g., Ascaris, (intestinal roundworm) and Trichinella (Whipworms)); Filarioidea (Loa boa filariasis, River Blindness (e.g., caused by Wucheria bancrofti, Brugia, and Onchocerca), and Dirofilaria immitis (heartworm)); Dracunculus (Guinea worm); Strongyloides (hookworms and pinworms); Flukes (including in the blood and/or liver); Tapeworms; Schistosomes; and plant nematodes including root knot, soybean cyst nematode, stem and bulb nematode, pine wood, foliar nematode, and seed gall. The polyanhydride particles are particularly effective for treating conditions such as River Blindness (Onchocerciasis) and Lymphatic Filariasis (Elephantiasis). The delivery via polyanhydride particles kills parasites at significantly lower concentrations than soluble delivered drugs.

Antiparasitic Polyanhydride Particles.

The polyanhydride particles can incorporate a wide variety of cargo molecules into the matrix of the polyanhydride polymers that make up the particles. The particles can incorporate one or more antiparasitic agents and one or more antibacterial agents. The antiparasitic agent can be an antihelminthic active agent. Antihelminthics are drugs that kill or expel parasitic worms (helminths) and other internal parasites from the body. They can act by either stunning or killing the parasite without causing significant damage to the host.

Antiparasitic agents that can be incorporated into the particles include antihelminthics and related active agents such as ivermectin, abamectin, albendazole, amphotericin B, artimisinin, auranofin, chloroquine, diethylcarbamazine, eflornithine, emetine, halofantrine, mebendazole, mefloquine, metronidazole, miltofosine, moxidectin, piperazine, praziquantel, primaquine, proguanil, pyrantel pamoate, quinine, quinolones, rapamycin, spiramycin, suramin, thiabendazole, tinidazole, and the like. The particles can include one of the aforementioned antiparasitic agents or a combination thereof.

Antibiotics that can be used in the particles include amikacin, bacitracin, carbapenem, ceftiofur, chloramphenicols, ciprofloxacin, clindamycin, cycloserine, doxycycline, erythromycin, ethambutol, fluoroquinolones, gentamicin, isoniazid, rifampin, streptogramin, streptomycin, tetracycline, vancomycin, and the like. The particles can include one of the aforementioned antibiotics or a combination thereof.

Loading percentage of active agents have been successful up to about 35% w/v of copolymer, and higher loading percentages, as high as 50%, can be achieved by techniques such as sequential loading of additional active agent and anhydride polymers to form larger particles (up to 5 μm or 10 μm in diameter). The particles can readily be loaded to about 0.1 wt. % to about 30 wt. % of total active agents. Standard loading of actives, or combinations of actives, is about 1 wt. % to about 20 wt. %, or about 5 wt. % to about 15 wt. %, or about 5 wt. % to about 10 wt. %.

Antimicrobial and Antiparasitic Activity.

To determine the antimicrobial and/or antiparasitic activity of the PANs, the release kinetics from encapsulating nanoparticles can be determined by quantifying the amount of encapsulated active agent, such as doxycycline, released from nanoparticles. The chemical structure of released active agent can be confirmed by analyzing for the presence of derivative molecules by circular dichroism and MS-HPLC. Activity for the released active agent can also be confirmed. Results of this analysis can be used to determine the highest amount of encapsulated active agent that can be delivered while remaining below host cytotoxicity levels for released active agent. This information allows for the determination of optimal antiparasitic or antimicrobial agent loading to develop whole animal treatment protocols.

### Pharmaceutical Formulations.

The compositions described herein, for example, the polyanhydride particles encapsulating two or more active agents, can be used to prepare therapeutic pharmaceutical compositions. The compositions described herein can be formulated as pharmaceutical preparations and can be administered to animal hosts, such as a mammalian host, for example a human patient. The preparation can be provided in a variety of forms. The forms can be specifically adapted to a chosen route of administration, e.g., oral or parenteral administration, intravenous, intramuscular, topical or subcutaneous administration, or administration by inhalation.

The pharmaceutical compositions may be systemically administered in combination with a pharmaceutically acceptable vehicle, such as an inert diluent or an assimilable edible carrier. For oral administration, compositions can be enclosed in hard or soft shell gelatin capsules, compressed into tablets, or incorporated directly into the food of a patient's diet. Compositions may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 1% to about 25% of the weight of a given unit dosage form. The amount of active compound in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active composition, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound in the particle composition may be further incorporated into sustained-release preparations and devices.

The active agent, e.g., the antimicrobial agent, antiparasitic agent, or combination thereof, may be administered in the polyanhydride particles intravenously or intraperitoneally by infusion or injection. Solutions of the pharmaceutical compositions can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the compositions adapted for the extemporaneous preparation of sterile injectable or infusible solutions or dispersions. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it may be advantageous to include isotonic agents, for example, sugars, buffers, or sodium chloride. Prolonged absorption of injectable compositions can be brought about by agents delaying absorption, for example, aluminum monostearate and/or gelatin.

Sterile injectable solutions can be prepared by incorporating the compositions in the desired amount into an appropriate solvent, optionally with various other ingredients enumerated above, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions.

For topical administration, compositions may be applied in pure form, e.g., in conjunction with a single carrier. However, it will generally be desirable to administer the active agents in the particles to the skin as a composition or formulation, for example, in combination with a dermatologically acceptable carrier formulation, such as a gel, ointment, lotion, foam, or cream.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which the compositions can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user. Examples of dermatological compositions for delivering active agents to the skin are known to the art; for example, see Jacquet et al. (U.S. Pat. No. 4,608,392), Geria (U.S. Pat. No. 4,992,478), Smith et al. (U.S. Pat. No. 4,559,157), and Wortzman (U.S. Pat. No. 4,820,508). Such dermatological compositions can be used in combinations with the compositions described herein.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a composition required for use in treatment will vary not only with the particular active and encapsulating polymer, but also with the route of administration, the nature of the condition being treated, and the age and condition of the patient, and will be ultimately at the discretion of an attendant physician or clinician.

The compositions can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The invention therefore provides therapeutic methods of treating parasitic infections in a mammal, which methods involve administering to a mammal having a parasitic infection an effective amount of a composition described herein. A mammal includes a primate, human, rodent, canine, feline, bovine, ovine, equine, swine, caprine, bovine and the like.

The ability of a compositions of the invention to treat parasitic infections may be determined by using assays well known to the art. For example, the design of treatment protocols, toxicity evaluation, data analysis, quantification of cell kill, and the biological significance of the use of screens are known and may be used in conjunction with the administration of the therapeutic particles described herein.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Examples

### Example 1

### Preparation of Polyanhydride Nanospheres and Microspheres

Sebacic acid (99%), 4-hydroxybenzoic acid, 1-methyl-2-pyrrolidinone anhydrous (99.5%), 1,6-dibromohexane (98.5%) and fluorescein-isothiocyanate-dextran (FITC-dextran) were purchased from Sigma-Aldrich (Milwaukee, Wis., USA). Other chemicals were purchased from Fisher Scientific (Pittsburgh, Pa., USA) and used as received.

Synthesis of SA and CPH pre-polymers and copolymers was performed as previously described (M. J. Kipper et al., Biomaterials. 23(22):4405-4412 (2002); E. Shen et al., J. Control. Release. 82(1):115-125 (2002); A. Conix, Poly[1,3-bis(p-carboxyphenoxy)propane anhydride], Macromolecular Synthesis, 2:95-98 (1966); and U.S. Pat. No. 7,659,322 (Vogel et al.); each incorporated herein by reference).

The resulting polymers were characterized using ¹H nuclear magnetic resonance to verify polymer chemistry, gel permeation chromatography to analyze molecular weight, and differential scanning calorimetry to determine glass transition temperature and crystallinity. All properties evaluated showed that the synthesized polymers were within accepted ranges.

Nanosphere Fabrication and Characterization.

FITC-dextran loaded nanospheres were fabricated by polyanhydride anti-solvent nanoencapsulation, similar to the method reported by Mathiowitz et al. for poly (fumaric acid-co-sebacic acid) polymers (E. Mathiowitz et al., Nature, 386(6623):410-414 (1997)). Active agents can be encapsulated into the nanospheres in a similar manner. Polymer (145.5 mg) was dissolved in methylene chloride (5 mL) held at room temperature for poly(SA) and 20:80 CPH:SA, and at 0° C. for 50:50 CPH:SA. FITC-dextran (4.5 mg) was added to the polymer solution and homogenized at 30,000 rpm for 30 seconds to create a suspension. The polymer/fluorescein solution was rapidly poured into a bath of petroleum ether at an antisolvent to solvent ratio of 80:1 held at room temperature (˜23° C.) for poly(SA) and 20:80 CPHSA, and −40° C. for 50:50 CPHSA (due to the lower glass transition temperature for 50:50 CPH:SA).

Polymer solubility changes due to the presence of anti-solvent caused spontaneous particle formation. These particles were removed from the anti-solvent by filtration (by aspiration using a Buchner funnel and Whatman #2 filter paper) and then dried overnight under vacuum. The procedure yielded a fine powder with at least 70% recovery. The nanosphere morphology was investigated using scanning electron microscopy (JEOL 840A, JEOL Ltd., Tokyo, Japan). Particle diameter was determined using quasi-elastic light scattering (Zetasizer Nano, Malvern Instruments Ltd., Worcester, UK).

### Examples of Polyanhydride Microspheres and Nanospheres

In various embodiments, polyanhydride microspheres and nanospheres can be copolymerized particles (“copolymer”) of a hydrophobic monomer (CPH) and a hydrophilic monomer (either SA or CPTEG). For example, certain polyanhydride nanospheres described herein are based on the monomers sebacic acid (SA), 1,6-bis(p-carboxyphenoxy)hexane (CPH), and/or 1,8-bis(p-carboxyphenoxy)3,6-dioxaoctane (CPTEG). These polyanhydride particles can be used for applications such as drug delivery for the treatment of River Blindness and Lymphatic Filariasis.

The polyanhydride particles that include CPH have hydrophobic properties, they resists hydrolytic degradation in vivo, and they resist degradation at acidic pH. They also degrade slowly, and the degradation occurs by a surface erosion mechanism (vs. bulk erosion by PLGA). Encapsulated cargo can thus be slowly released during surface erosion. Very little leaching of cargo from intact particles occurs. The relative degradation in tissues is weeks to months, depending on the size and specific polyanhydride used to prepare the particles.

### Example 2

### Polyanhydride Nanoparticle Delivery Platform Enables Enhanced Killing of Filarial Worms

Within the last decade there has become an increasing concern regarding the ability to effectively control and eradicate current infections of the filarial endoparasite B. malayi, B. pahangi, and its symbiotic bacteria Wolbachia. Filarial diseases represent a significant social and economic burden in areas that are endemic with lymphatic filariasis (LF) and river blindness (RB). This example demonstrates the use of an amphiphilic polyanhydride (AmPa) nanoparticle-based platform for the co-delivery of doxycycline to the symbiotic bacteria, Wolbachia, and the antiparasitic, ivermectin, to target the adult worm.

Co-encapsulation within AmPa nanoparticles increased drug efficiency in killing adult male and female B. malayi worms by 40-fold and reduced microfilaria shedding by females over 4 fold. Time to death of the macrofilaria was also significantly reduced in AmPa+IVM/Doxy treated groups. Visualizing treated worms by confocal microscopy revealed intact nanoparticles distributed throughout the deeper tissues of the worm as early as 5 hours, coinciding with cessation of worm movement. The mechanism can occur via the ability of the AmPa nanoparticles to penetrate the cuticle and outer dermis of the B. malayi worm to rapidly deliver high, local concentrations of drugs directed at the parasite and endosymbiotic bacteria. These findings have significant beneficial consequences for reducing the amount of drug, length of treatment and undesired side-effects associated with therapies against filarial infections.

Materials and Methods.

Acquisition and Storage of B. malayi and Microfilariae (MF).

Live adult B. malayi females, males and microfilariae were acquired from the Infectious Disease (NIH/NIAID) Filariasis Research Reagent Repository Center (FR3) (University of Georgia, Athens, Ga.). Adult worms where maintained in Roswell Park Memorial Institute (RPMI) 1640 medium supplemented with 10% fetal bovine serum (FBS) and 1% penicillin-streptomycin. The B. malayi were held in an incubator at a temperature of 37° C. supplemented with 5% carbon dioxide (CO₂). Female and male worms were stored individually in 48 well microtiter plates containing 1 mL of RPMI-1640. Previously shed microfilariae were housed in 50 mL conical tubes containing 25 mL of RPMI-1640. Upon arrival of the filarial worms they were separated and placed into individual 48 well microtiter plates that contained the previously described media.

Motility Scoring.

The motility scoring of the adult worms where monitored utilizing a 2× objective on a Nikon Microscope with observations over a 30 second period of time and were scored utilizing the following 0-5 scoring system.

| Score | Motility |
|---|---|
| 5 | 0% motility reduction: head and tail uninhibited as well as |
| | mid-section unaffected |
| 4 | 1-25% ability to visualize both the head and tail movements |
| | easily as well as midsection |
| 3 | 26-49% reduction showed a partial mid-section paralysis |
| 2 | 50-74% reduction showed full mid-section paralysis followed |
| | by a substantial reduced movement in the head and tail |
| 1 | 75-99% reduction showed full mid-section paralysis followed |
| | by either head and or tail paralysis but not limited to |
| | occasional movement over a 30 second time period |
| 0 | 100% as dead and non-motile |

The loss of motility of adult worms was compared with that of respective untreated controls.

### Synthesis of Ivermectin and Doxycycline-Loaded Polyanhydride Nanoparticles.

Materials.

For the synthesis of the CPH and CPTEG monomers, the polyanhydride polymers, 20:80 CPH:SA and 20:80 CPTEG:CPH, and the ivermectin and doxycycline-loaded polyanhydride nanoparticles, acetic acid, acetic anhydride, acetone, acetonitrile, chloroform, dimethyl formamide, ethyl ether, hexane, methylene chloride, pentane, petroleum ether, potassium carbonate, sodium hydroxide, sulfuric acid, and toluene were purchased from Fisher Scientific (Fairlawn, N.J.). The chemicals 1,6-dibromohexane, 1-methyl-2-pyrrolidinone, hydroxybenzoic acid, N,N-dimethylacetamide, sebacic acid, and tri-ethylene glycol were obtained from Sigma Aldrich (St. Louis, Mo.). The chemical 4-para-fluorobenzonitrile was purchased from Apollo Scientific (Cheshire, UK). For ¹H NMR analysis deuterated chloroform and deuterated dimethyl sulfoxide were purchased from Cambridge Isotope Laboratories (Andover, Mass.). A fluorescent dye, Rhodamine B, was purchased from Sigma Life Science (St. Louis, Mo.). Ivermectin and doxycycline were also purchased from Sigma Life Science (St. Louis, Mo.).

Polymer Synthesis.

Synthesis of the monomers used, 1,6-bis(p-carboxyphenoxyhexane) (CPH) and 1,8-bis(p-carboxyphenoxy)-3,6-dioxaoctane (CPTEG), was performed as described by Torres, Vogel, Narasimhan, and Mallapragada (J. Biomed. Mater. Res. A 76, 102-110 (2006)). The 20:80 molar copolymers of CPTEG and CPH, and CPH and SA, were synthesized through melt condensation polymerization, as described above in Example 1, or by the methods of Kipper et al. (Biomaterials 23, 4405-4412 (2002)). Molecular weight was confirmed using ¹H NMR.

Nanoparticle Fabrication.

Doxycycline and ivermectin-loaded nanoparticles of 20:80 CPH:SA and 20:80 CPTEG:CPH were fabricated through solid/oil/oil nanoprecipitation, as described above in Example 1, or by the methods of Kipper et al. (Biomaterials 23, 4405-4412 (2002)). For example, ivermectin and doxycycline, each at 5% (w/w), with Rhodamine B at 2% (w/w) were added to the polymer. The solid drug and polymer mixture was then dissolved in methylene chloride at a concentration of 20 mg/mL, followed by rapid precipitation into the antisolvent, pentane. The polyanhydride nanoparticles were then collected using vacuum filtration. The drug-loaded particles were characterized for size and morphology by scanning electron microscopy (SEM, FEI Quanta SEM, Hillsboro, Oreg.).

Effects of drugs on adult stages of B. malayi.

Ivermectin (22,23-dihydroavermectin B1) and doxycycline hyclate (C₂₂H₂₄N₂O₈ HCl 0.5H₂O 0.5C₂H₆O) were purchased from Sigma Life Science and dissolved in DMSO (final concentration of 0.02 v/v). RPMI-1640 medium was prepared such that the final drug concentrations (in μM/mL) were 195, 49, 10, 5 and 40 nM. Control medium contained 0.02% DMSO but no drug. Individual male and female adult worms that were previously placed into 48 well flat bottom culture plates upon arrival had new fresh media that contained 1 mL of RPMI-1640, 0.01% Streptomycin/Penicillin and 10% fetal calf serum. B. malayi incubated at 37° C. and 5% CO₂ for 1-2 hours for acclimatization to evaluate base line motility before experiment began. Encapsulated chemistry compounds and standard antifilarials where added as described above. Controls received equal amounts of media but lacked the PANs and standard antifilarials. B. malayi were incubated at 37° C. and 5% CO₂ and monitored at hourly intervals as indicated for changes in motility.

Effects of Drugs on B. malayi and Microfilariae.

Previously shed microfilariae (MF) where obtained from the shipping media along with any MF that were shed over the first 1-2 hours from females housed in the 48 well plates. Conical tubes were placed into a centrifuge and spun down for ten minutes at 200 rpms. Old media was then removed and replaced with the above described 1640-RPMI media. Microfilariae were randomly divided into control and treatment groups that contained approximately 1500 microfilariae per group was estimated based on a single well 14.80 mm² grid plate.

Motility was monitored at indicated hourly intervals and scored following observing worms using an inverted, phase-contrast Nikon microscope with a 10× objective. An average of 5 Fields Of View (FOV) that contained 2 grid sections were used to calculate score for each treatment group and time point. Motility scoring was recorded based on the number of times a microfilaria bent its body such that the anterior and posterior ends met was considered one full body movement. The number of times a microfilaria contracted its body within one minute was recorded. The scoring technique utilized is that which was described previously for adults. We utilized approximately 1500 microfilariae per treatment group and results presented are the average of three separate experiments.

Effects of Drugs on B. malayi Shedding.

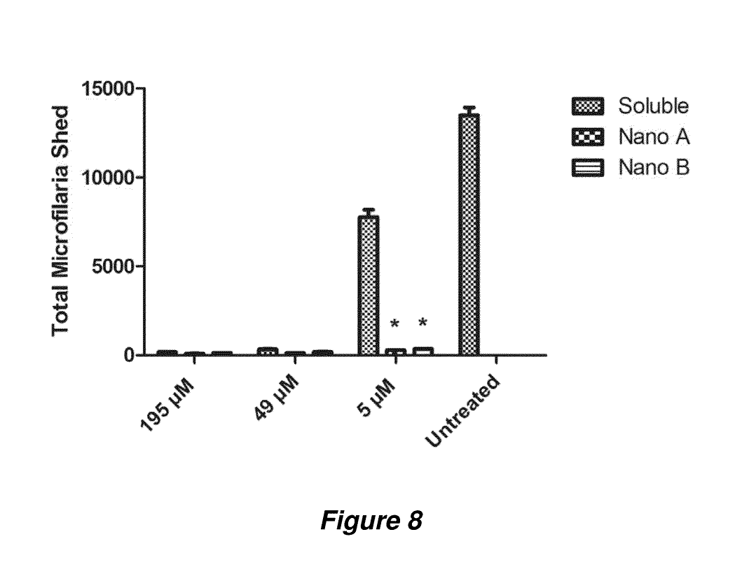

Concurrently with the adult motility assay adult females were monitored on the effectiveness of the above drugs ability to decrees MF shedding as well as the motility of those shed during the experiment. Adult females were given a scoring system similar to that of the motility in regards to the number of MF shed. Shedding calculations where obtained by a 10× Nikon microscope utilizing a 5 FOV utilizing the same grid system as previously described. The 5 FOV where then averaged to obtain the total MF shed per adult female. The following 0-3 scoring system was used. 0=0 shed; 1=1-50 MF shed; 2=51-100 MF shed; and 3=100+ MF shed. Shedding was monitored visually and the number of microfilaria was recorded by treatment group over 10-14 days (see FIG. 8). The limit of detection for each day was 1200 MF, and the motility of MF shed was monitored based on the previous motility scoring system.

Florescent Microscopy of B. malayi.

Once complete cessation had occurred adult B. malayi males and females were washed twice utilizing phosphate-buffered saline (PBS), then immediately transferred for fixation into 4% paraformaldehyde for 1 hour and then washed again two times before individual worms were mounted on microscope slides with Prolong Gold (Life Technologies). Slides were then stored at 4° C. Images were examined by laser-scanning confocal microscopy (LCM) (Fluoview, Olympus). Anatomical subunits as well as presence of AMPs were localized by confocal laser-scanning microscopy, with counterstaining against nuclei. Counterstaining distinguished the major anatomical features of B. malayi including the oral opening, the nerve ring, the ES apparatus, the inner body, esophageal track and anal pore.

Results.

Encapsulation of Doxycycline and Ivermectin into PANs Provides Sustained Drug Release.

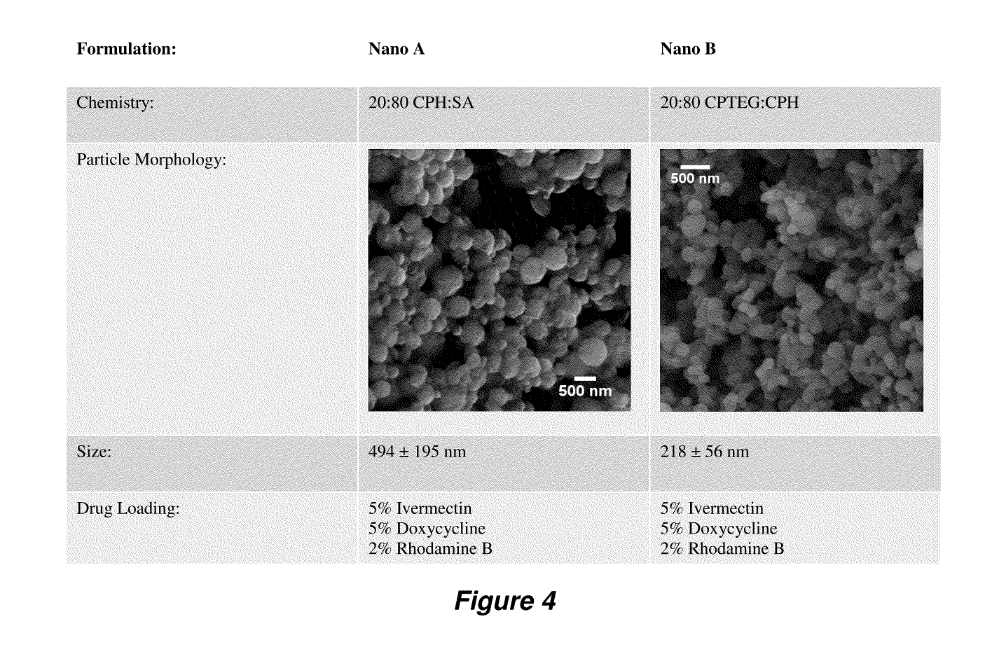

As summarized in FIG. 4, doxycycline and ivermectin were encapsulated into polyanhydride nanoparticles at a 5% (w/w) loading for each drug. Additionally, a fluorescent dye, Rhodamine B, was optionally co-encapsulated with the drugs at a 2% (w/w) loading to track the interactions of the nanoparticles with the B. malayi macrofilaria through confocal microscopy.

The active agent payload was encapsulated into two polyanhydride nanoparticle chemistries: a 20:80 molar copolymer of 1,6-bis(p-carboxyphenoxy)hexane and sebacic acid (“20:80 CPH:SA” or Nano A), and a 20:80 molar copolymer of 1,8-bis(p-carboxyphenoxy)-3,6-dioxaoctane and 1,6-bis(p-carboxyphenoxy)hexane (“20:80 CPTEG:CPH” or Nano B). After encapsulation of the anti-filarial drugs by nanoprecipitation, the surface morphology and size was examined using scanning electron microscopy. Surface morphology was found to be consistent with previous work and the scanning electron photomicrographs are shown in FIG. 4. Additionally, particle size was analyzed, and also found to be consistent with our previous work (Ulery et al., Pharm. Res. 26, 683-690 (2009); Petersen et al., Acta Biomater. 6, 3873-3881 (2010)). The average size for the 20:80 CPH:SA, or Nano A, nanoparticles was found to be 494±195 nm, and the average size for the 20:80 CPTEG:CPH nanoparticles, Nano B, slightly smaller at 218±56 nm.

Using high-performance liquid chromatography, the release kinetics of ivermectin and doxycycline from the PANs was able to be quantified, FIG. 3. Comparing the release kinetics of the two polyanhydride chemistries indicates a larger initial burst and faster release of the doxycycline from the Nano A formulation, 20:80 CPH:SA. This is as expected based on the polymer chemistry, and consistent with previous release kinetic profiles from PANs. Sebacic acid (SA) is less hydrophobic than 1,6-bis(p-carboxyphenoxy)hexane (CPH), thus the higher SA content in the Nano formulation leads to a larger burst, and an overall faster release profile of the doxycycline. In contrast the Nano B formulation, 20:80 CPTEG:CPH has an 80 molar percent CPH content, making this a more hydrophobic polymer, leading to a smaller initial burst. It was also observed that there was a much larger early burst release of the doxycycline from both PAN chemistries as compared to the release kinetics of the doxycycline.

Based on the respective chemistries of our two formulations, which both have an uneven distribution of each of the copolymers, microphase partitioning of the two copolymers can occur. The hydrophobic drug, ivermectin, may be preferentially partitioning to the CPH-rich domains of each formulation, leading to the slow release profile observed for both the Nano A and Nano B formulations.

Drug Delivery Using PANs Significantly Increases B. malayi Macrofilaria Mortality.

The effectiveness of the PANs when directly compared to soluble treatment shows the overall percent survival (Table 1) for B. malayi over the duration of the study period with a single treatment of the groups described above. The results clearly show the superior effectiveness of Nano A and Nano B throughout all treatment groups when compared to the soluble group.

TABLE 1
Summary of mean time of death in hours with total numbers of dead
worms at the conclusion of the study interval (2 weeks). Total numbers
of dead worms, most notably at lower doses, is greater in PANs +
drug treated experiments compared to soluble drug alone.

| | B. malayi Females | | B. malayi Males | |
|---|---|---|---|---|
| | Avg. | #of | Avg. | #of |
| Dose | TOD | Deaths | TOD | Deaths |
| 195 μM IVM/Doxy Soluble | 4.5 | 5/5 | 5 | 2/5 |
| 195 μM Formulation A | 0.51 | 5/5 | 0.67 | 5/5 |
| 195 μM Formulation B | 0.34 | 5/5 | 0.36 | 5/5 |
| 49 μM IVM/Doxy Soluble | 7.2 | 1/5 | 6.8 | 2/5 |
| 49 μM Formulation A | 1.6 | 5/5 | 2.2 | 5/5 |
| 49 μM Formulation B | 2.43 | 5/5 | 3.7 | 5/5 |
| 10 μM IVM/Doxy Soluble | 12 | 0/5 | 8.3 | 2/5 |
| 10 μM Formulation A | 1.49 | 5/5 | 4 | 5/5 |
| 10 μM Formulation B | 1.88 | 5/5 | 4.2 | 5/5 |
| 5 μM IVM/Doxy Soluble | 15 | 0/5 | 10 | 0/5 |
| 5 μM Formulation A | 4.57 | 5/5 | 6 | 5/5 |
| 5 μM Formulation B | 3.86 | 5/5 | 5.25 | 5/5 |

Formulation A: 20:80 CPH:SA (Nano A);
Formulation B: 20:80 CPTEG:CPH (Nano B).

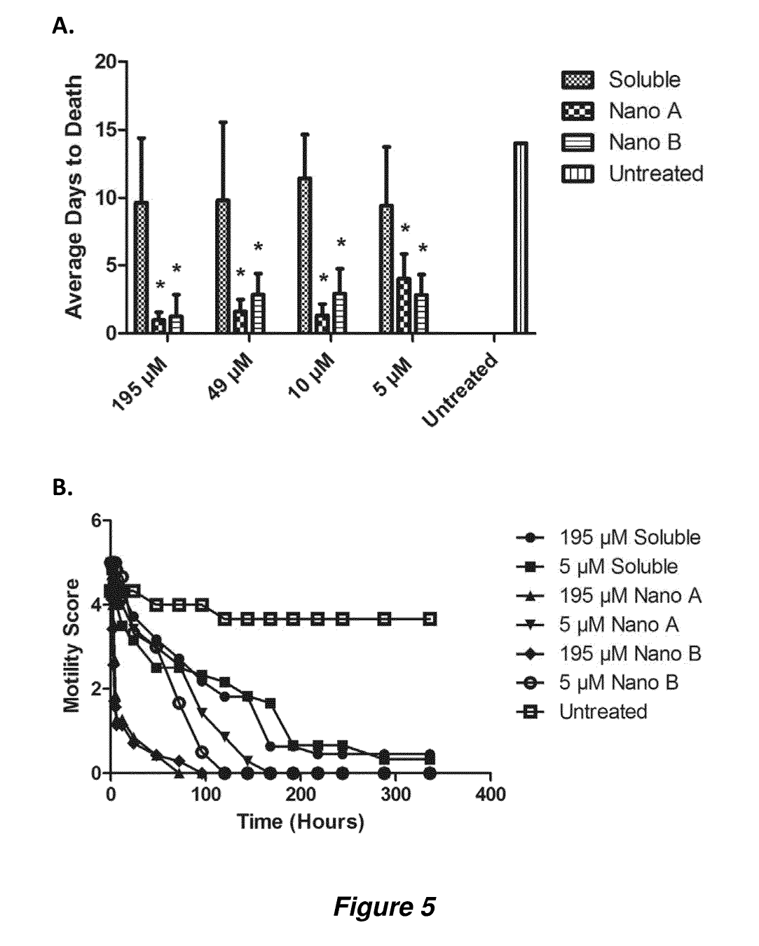

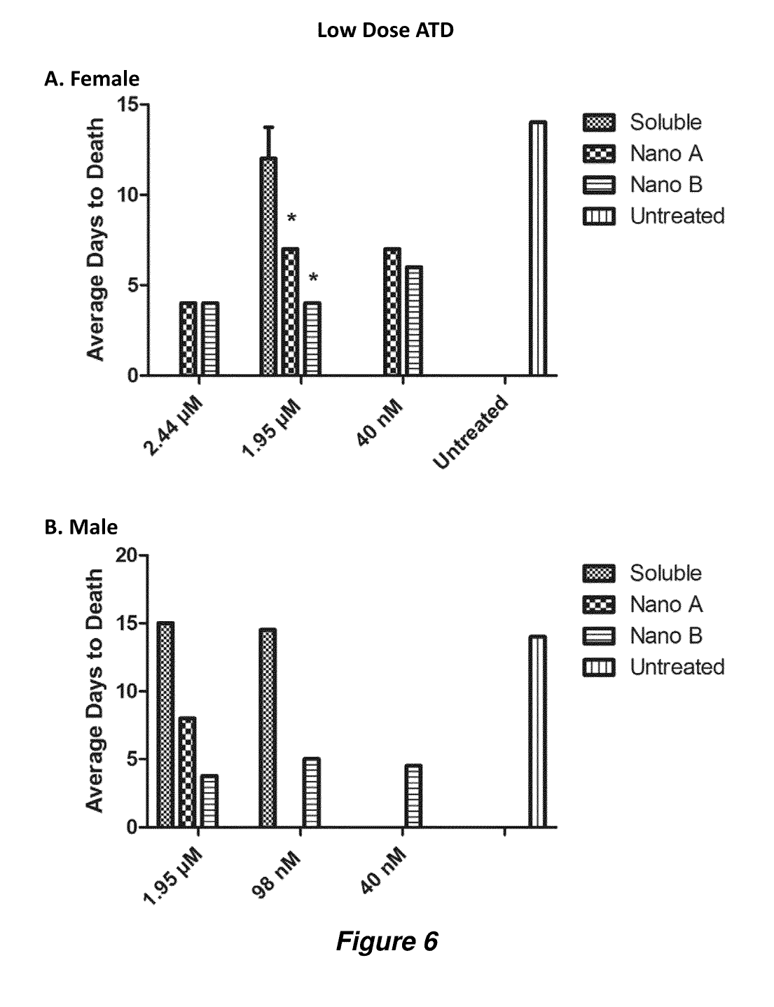

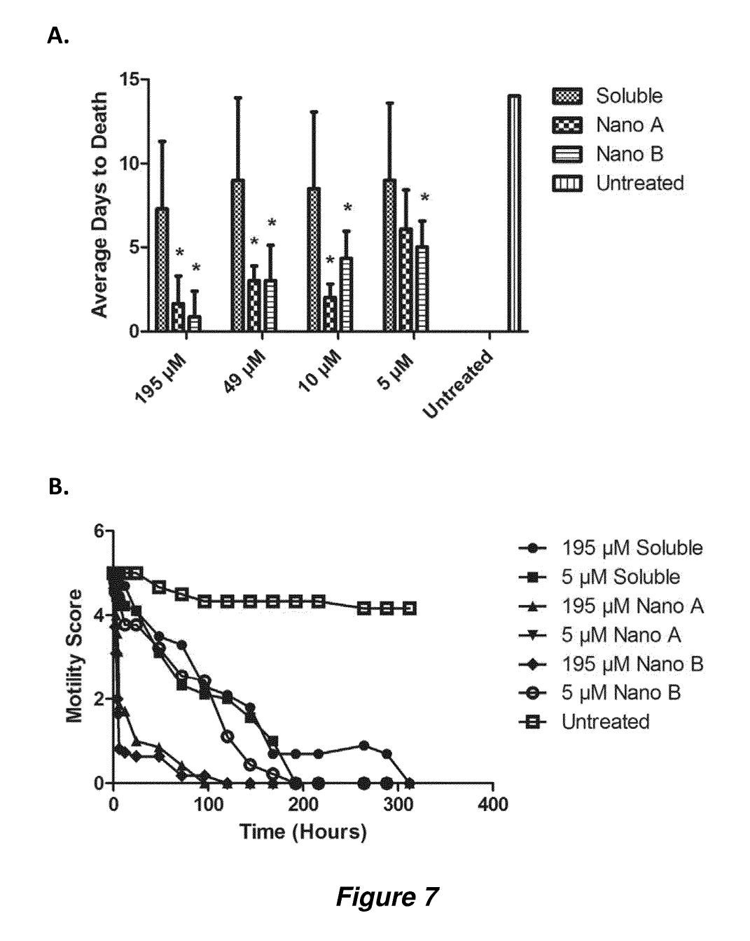

To show the differences between the effectiveness soluble vs. PANs compositions of dual ivermectin/doxycycline on average days to death, we conducted a fourteen day in vitro assay that shows the average days to death of B. malayi females (FIG. 5A and FIG. 6A) and B. malayi males (FIG. 6B and FIG. 7A). For the females we were able to show effectiveness of Nano A and Nano B having had a significant difference (p<0.001) in the overall combined time to death when compared directly to soluble dual IVM/Dox. Treatment groups ranged from N=4-13, respectively. The dual soluble treatment group of 195 μM had an average time to death being greater than nine days and an average death of 63% of the individuals (Table 1), whereas the average time to death of the Nano A and Nano B groups was less than 1.2 days, with 100% death of the worms. At the lowest treatment dose of 5 μM the soluble IVM/Doxy average time to death was greater than 9 days with only a 14% death rate (Table 1). In contrast the Nano A and Nano B groups have an average time to death of four days or less, with 100% macrofilarial death.

For the B. malayi males (FIG. 7A) Nano B showed a significant difference (p<0.001) but at the lowest concentration tested, the Nano A group was not significantly different than the soluble treatment group. Treatment groups ranged from N=4-16, respectively. The dual soluble treatment group of 195 μM had an average time to death being greater than seven days and an average death 50% (Table 2), whereas the average time to death of the Nano A and Nano B groups was less than 1.6 days, with 100% death. At the lowest treatment dose of 5 μM the soluble IVM/Doxy average time to death was greater than 9 days and had a death rate of 30%, whereas the Nano A and Nano B groups have an average time to death of six days or less with 100% death of the parasites.

In conjunction with the average days to death assay we also monitored the motility over time to help determine the overall effectiveness of the PANs (FIG. 5B and FIG. 7B). Treatment groups ranged N=4-13, respectively. The figures summarize a direct scoring of motility as previously described. We were able to determine that with the female B. malayi there is a significant decline in motility with the 195 μM and 5 μM groups and the experiments showed an exponential reduction of overall motility of the Nano A and Nano B groups when directly compared to the soluble groups. As observed with the females, a very similar trend was observed with the adult male B. malayi (FIG. 7B). Treatment groups ranged N=4-16, respectively. The figures summarize a direct scoring of motility as previously described above. The figure depicts 195 μM and 5 μM groups and shows an exponential reduction of overall motility of the Nano A and Nano B groups when directly compared to the soluble groups.

We also sought to determine the extent that a dosing strategy could be reduced while still maintaining increased efficacy (FIG. 6). We showed that the overall effectiveness of Nano A and Nano B provided a significant difference (p<0.001) in the overall combined time to death when compared directly to soluble dual IVM/Dox at the treatment level of 1.95 μM concentration. The 1.95 μM treatment group shows how significantly effective the PANs are at actively killing B. malayi compared to soluble drugs. This concentration shows a 100 fold reduction in the amount of drugs that are necessary to effectively kill the parasites. Treatment groups ranged from N=3-5, respectively. The dual soluble treatment group of 1.95 μM had an average time to death being greater than twelve days and an average death of 10% (Table 2), whereas the Nano A and Nano B groups had an average time to death of less than 6.4 days, with 100% macrofilarial death.

TABLE 2
Survival of B. malayi Female Worms: NP
Formulations and Dose Response: Nano A.

| Formulation | # Treated | % Death |
|---|---|---|
| 195 μM Soluble IVM/Dox | 11 | 64% |
| 195 μM NP-IVM/Dox | 13 | 100% |
| 49 μM Soluble IVM/Dox | 5 | 40% |
| 49 μM NP-IVM/Dox | 5 | 100% |
| 10 μM Soluble IVM/Dox | 7 | 43% |
| 10 μM NP-IVM/Dox | 4 | 100% |
| 5 μM Soluble IVM/Dox | 7 | 14% |
| 5 μM NP-IVM/Dox | 7 | 100% |

Encapsulation into PANs Reduces Microfilarial Shedding.

To determine if the decreased killing time of the adult females had a positive effect on reduction of shedding of microfilariae observed in conjunction with the adult in vitro assays as described above, we determined the total overall number of microfilaria shed over the duration of fourteen days (FIG. 8) with a limit of detection of 1200 MF per day. Both the soluble and Nano A and Nano B groups were significant at reducing the overall MF shed at the high dose of 195 μM concentration (a concentration unlikely to be practically achieved in vivo), but as we reduced the concentration of IVM/Doxy a significant difference (p<0.001) was observed when comparing the IVM/Doxy soluble groups to that of Nano A and Nano B groups.

Microfilaria Motility.

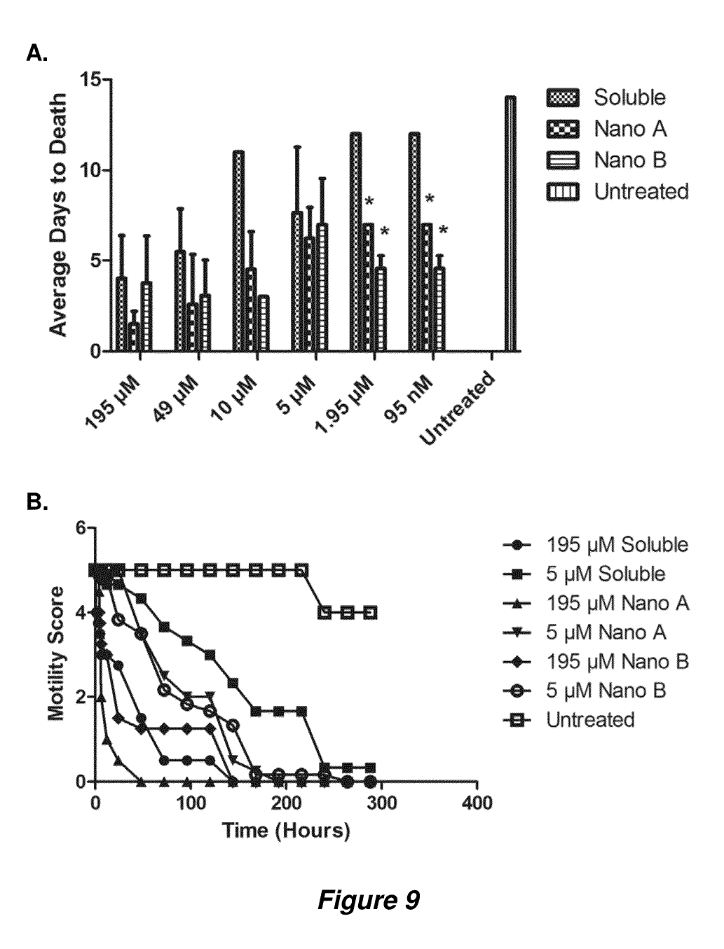

The effects of the soluble and PANs dual ivermectin/doxycycline treatment groups on motility of B. malayi microfilariae (FIG. 9) were observed for seven days with N=600 for each experiment (repeated 3 times). Similar to the effects observed for adults, the average days to death as well as the motility of soluble drug when compared to Nano A and Nano B treated microfilariae differed significantly (p<0.0001) at the lower concentrations of 10 and 5 μM. The PANs groups were more effective at all concentrations.

PAN Delivery Vector Increased Permeability of Fluorescent Dye into Microfilaria.

Confocal imaging (FIG. 2) of female B. malayi with soluble IVM/Doxy with a Rhodamine Red (Rho) florescent marker and Nano A group also with a Rho florescent marker was analyzed to observe the distribution/penetration/absorption comparisons between the two groups. The inability of the Rho florescent marker of the soluble treatment to adequately penetrate the outside cuticle of the worm is clearly evident when compared to that of the Nano A group, which has clear distribution/penetration/absorption. This image is a positive indicator that shows the effectiveness and a mode of action of the PANs, directly towards B. malayi.

Conclusions.

This example demonstrates the use of a polyanhydride nanoparticle-based drug delivery platform for the co-delivery the antiparasitic drug ivermectin to reduce the macro- and microfilarial burden and the antimicrobial doxycycline to eliminate the symbiotic bacteria, Wolbachia. The co-delivery of doxycycline and ivermectin in polyanhydride nanoparticles (PANs) effectively killed adult B. malayi filarial worms with up to a 100-fold reduction in the amount of drug used. Furthermore, the time to death of the macrofilaria was significantly reduced when the anti-filarial drug cocktail was delivered via PANs. The PANs interact and adhere with the cuticle, penetrate the outer membrane, delivery of the patristic drugs to vital areas within B. malayi worm. The PANs are also consumed orally by the parasites, thereby providing a second delivery mode to the internal tissues of the parasites. The combined modes of delivery were found to deliver drugs at high enough microenvironment concentrations to cause death significantly faster than soluble drug treatment. These findings provide a method to reduce the amount of drug and the length of time required for treating parasitic infections.

### Example 3

### Amphiphilic Polyanhydride Nanoparticles Enable Increased Killing of Brugia malayi and Brugia pahangi

Amphiphilic polyanhydride nanoparticles (PANs) are solid, surface eroding particles, that encapsulate cargo such as small molecules or proteins, which cargo becomes an integral component of the particles. This next generation platform can be used as a multiple-drug delivery system with the ability to increase the efficacy of a drug and its interaction with parasites. Amphiphilic polyanhydride nanoparticles (PANs) having a variety of chemistries (e.g., 20:80 CPH:SA; 20:80 CPTEG:CPH; and 50:50 CPTEG:CPH) can be prepared with a combination of antiparasitic agents and antibiotic agents to provide a lethal delivery vehicle for the parasites that cause parasitic infections. PANs loaded with an antiparasitic and an antibiotic were found to provide enhance killing of B. malayi and B. pahangi compared to a combination of the corresponding soluble drugs.

We sought to use these properties of PANs to exploit the dependence of filarial worms on their symbiotic bacteria Wolbachia by developing a nanoparticle therapy to co-deliver an antiparasitic drug (ivermectin) with the antibacterial drug (doxycycline). In vitro studies with the Brugia malayi demonstrate an increase in antiparasitic activity, as measured by recording worm motility following exposure to soluble or encapsulated drugs. The average time to death (TOD) observed in Brugia malayi females was reduced from 6 days for soluble 4.8 μg ivermectin/doxycycline to 18 hours for encapsulated drugs. The lowest dose of PANs (0.06 μg) matched the highest dose of soluble drugs (100 μg) TOD of 3 days, exhibiting similar dose response that is 1/1670 less drug when encapsulated. See Table 3.

TABLE 3
Greater Killing of Worms at Lower Doses of PANs. Summary of mean
time of death in hours with total numbers of dead worms at the
conclusion of the study interval (typically 2 weeks). Total numbers
of dead worms, most notably at lower doses, is greater in PANs +
drug treated experiments compared to soluble drug alone.

| | B. malayi Females | | B. malayi Males | |
|---|---|---|---|---|
| | Avg. | #of | Avg. | #of |
| Dose | TOD | Deaths | TOD | Deaths |
| 195 μM IVM/Doxy Soluble | 4.5 | 5/5 | 5 | 2/5 |
| 195 μM Formulation A | 0.51 | 5/5 | 0.67 | 5/5 |
| 195 μM Formulation B | 0.34 | 5/5 | 0.36 | 5/5 |
| 195 μM Formulation C | 0.9 | 5/5 | 1.4 | 5/5 |
| 49 μM IVM/Doxy Soluble | 7.2 | 1/5 | 6.8 | 2/5 |
| 49 μM Formulation A | 1.6 | 5/5 | 2.2 | 5/5 |
| 49 μM Formulation B | 2.43 | 5/5 | 3.7 | 5/5 |
| 49 μM Formulation C | 3.4 | 5/5 | 4 | 5/5 |
| 10 μM IVM/Doxy Soluble | 12 | 0/5 | 8.3 | 2/5 |
| 10 μM Formulation A | 1.49 | 5/5 | 4 | 5/5 |
| 10 μM Formulation B | 1.88 | 5/5 | 4.2 | 5/5 |
| 10 μM Formulation C | 2.04 | 5/5 | 4.5 | 5/5 |
| 5 μM IVM/Doxy Soluble | 15 | 0/5 | 10 | 0/5 |
| 5 μM Formulation A | 4.57 | 5/5 | 6 | 5/5 |
| 5 μM Formulation B | 3.86 | 5/5 | 5.25 | 5/5 |
| 5 μM Formulation C | 5.17 | 5/5 | 6.33 | 5/5 |

Formulation A: 20:80 CPH:SA;
Formulation B: 20:80 CPTEG:CPH;
Formulation C: 50:50 CPTEG:CPH.

Microscopy revealed rapid penetration of the worms by nanoparticles at 6 hours post-therapy, coinciding with a significant decrease in motility scores. Surface modification of nanoparticles had a negative impact on efficacy, indicating that direct surface interactions between the filaria and PANs are required. Similar results were obtained with adult B. malayi male worms and adult worms of both sexes of B. pahangi. Encapsulation of drug combinations into PANs will serve to repurpose current antifilarial treatment methods into more potent therapies offering greater patient compliance due to reduced number of doses and greater efficacy in endemic areas.

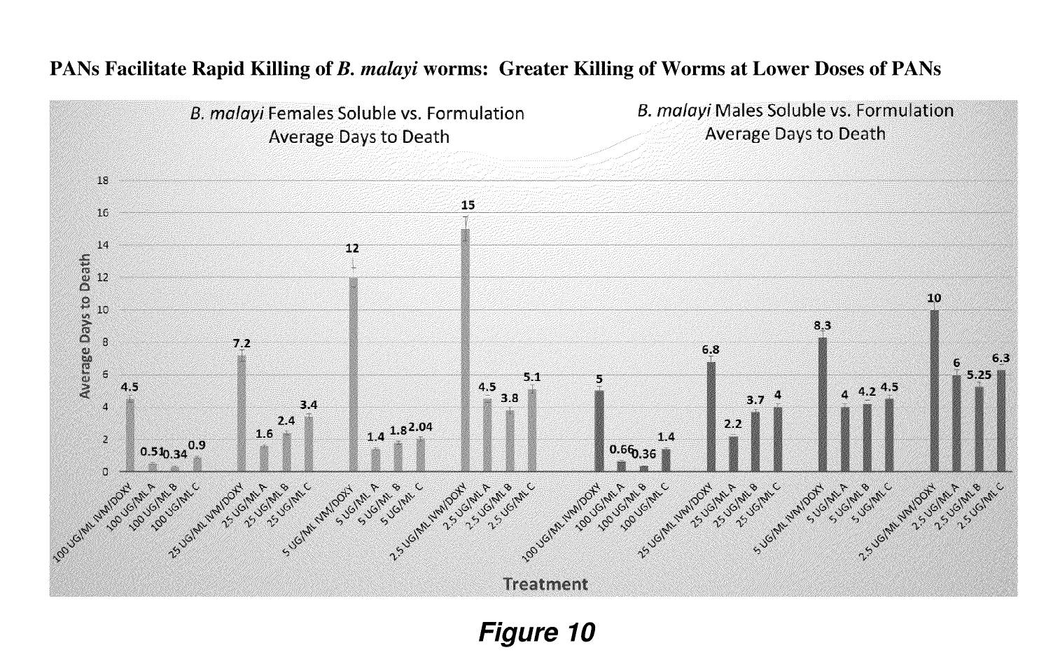

The ability of the PANs to slowly erode and release the cargo molecule in a controlled manner allows for specificity against both adult nematodes and the symbiotic bacteria Wolbachia. The results of this study show that the PANs described herein can interrupt the life cycle of the nematode, not by just reducing microfilaria load, but by directly increasing mortality in the adult population. By co-encapsulating ivermectin (IVM) and doxycycline into PANs, we have shown that the PANs can be a more effective route of drug administration due to the increased efficacy that is obtained between the direct interactions of the particles with the filarial nematode. Thus, the drug-encapsulating PANs provide a drug delivery platform able to increase efficacy and overall safety of current anti-parasitic therapies. See FIG. 10 for details and results.

Methods.
Female and male adult worms were acquired;
Separated into individual wells within 48 well plate in 1 mL RPMI 1640+10% FBS (1% Pen/Strep);
Began experiment the following morning;
Ivermectin in 2% DMSO/doxycycline in dH₂O;
Worms were scored for motility by visual inspection scale 0-5 (5 greatest motility vs. 0 no movement observed within 30 second time window);
Observations were recorded.

Results confirmed the development of a new drug delivery platform based on PANs and encapsulated antiparasitic and antibiotic drugs. Based on the in vitro results shown above, and in FIG. 10, the PANs provide the ability to increase efficacy by 167-1,667 times compared to soluble antibiotics in the same timeframe. These results indicate that the focused drug delivery by PANs provides substantial increases in efficacy with smaller total amounts of antibiotics. In summary, antiparasitic and antibiotic drugs within PANs consistently reduced the average time to death of adult B. malayi worms. Alternate drug combinations that replace ivermectin with other antiparasitic drugs and/or replace doxycycline with other antibiotic drugs can be similarly effective.

### Example 4

### Treatment of Neglected Tropical Diseases

Amphiphilic particle based drug delivery platform can dramatically improve the lives of people living in areas endemic with filarial diseases Lymphatic Filariasis (LF), River Blindness (RB) and Soil-Transmitted Helminth (STH) diseases. Improvements in efficacy and safety of therapies for neglected tropical filarial diseases are possible by using the biodegradable polyanhydride nanoparticle drug delivery platform described herein. The drug delivery system makes possible co-encapsulation of ivermectin with doxycycline in a single dose therapeutic that has the ability to provide delayed/persistent drug release, enhanced pathogen/organ targeting, and is compatible with multiple routes of administration (e.g., transdermal/transcutaneous, oral, parenteral, spray, and application to the soil in cases of infected plants).

| | LF | RB | STH |
|---|---|---|---|
| Source | Mosquito | Blackfly | Contaminated soil |
| Route | Bite | Bite | Bare feet |
| Host State | Lymphatics | Subcutaneous Skin | Blood-lung |
| Transient | | | |
| Chronic | Lymphatics | Skin & Eye | Gastrointestinal |
| Potential | SubQ micro/nano | SubQ micro/nano | Film adhesive; |
| Therapy | particles; | particles; | Gastric/Oral/ |
| | Direct site admin | Micro Eye drops | Intranasal |
| Additional | Dox: kills | Dox: kills | |
| therapy | Wolbachia | Wolbachia | |

Unconventional aspects nanoparticles and the methods described herein are that the pathogen-mimicking abilities of amphiphilic polyanhydride micro and nanoparticles that can be designed to slowly release anti-parasitic drugs, along with antibiotics such as doxycycline, to attack both juvenile (microfilaria) and adult filarial for preventative or therapeutic treatments.

The discovery that endosymbiotic bacteria Wolbachia is present within filarial worms that cause LF and RB presents an additional means to treat these diseases. These endosymbiotic bacteria provide a mutualistic benefit to the host worm. Antibacterial treatment with doxycycline improves clearance of the worm and works well in combination therapies with both ivermectin and albendazole, two frontline antiparasitic drugs. Adding doxycycline as a therapy presents logistical problems in that the antibiotic is cleared from the host more rapidly than the antiparasitic drugs. Multiple doses of doxycycline would be needed to provide the full therapeutic benefit. Given the single dosing every several months is typical for Mass Drug Administration (MDA) programs, it would be beneficial to incorporate an extended release doxycycline therapy with the chosen anti-parasitic drug in a single delivery vehicle.

### Effective Anti-Filarial and Helminth Therapies.

Particle Synthesis. Synthesize antiparasitic and antibiotic encapsulated polyanhydride nanoparticles and microparticles; quantify extended release of antibiotic in aqueous environments. Combinations of antiparasitic drugs can be co-delivered in a single particle; combinations of antibiotics can also be co-delivered in the same particle.

Safety. Quantify kinetics and amounts of released antibiotic within sera and tissues. Examine serum protein levels and tissue pathology for nanoparticle induced cytotoxicity.

In Vitro Efficacy. Quantify and compare antimicrobial activity of soluble active agents and active agent-loaded particles against Wuchereria, Brugia spp. and O. volvulus in vitro. Conditions can be varied and can include variations of broth culture and intracellular survival assays performed by infecting monocytes in tissue culture. Measure monocyte associated antibiotic concentration over infection period and beyond.

In Vivo Efficacy. Measure improved elimination of persistent Wuchereria, Brugia spp. and O. volvulus from experimentally infected guinea pigs and compared to treatment with soluble antibiotic.

Disease and Treatments

| Combinatorial Drug Particles | RB | LF | STH |
|---|---|---|---|
| Doxy + Ivermectin | YES | YES | — |
| Doxy + Albendazole | — | YES | YES |
| Doxy + Albendazole + Dec | YES | YES | YES |

Administration of the PANs can quantifiably lower the number of viable Wucheria, Brugia spp. and O. volvulus by FACS analysis recovered from broth culture, in vitro cell culture infection, and Wuchereria, Brugia spp. and O. volvulus persistence modeling in animals by the active agent-encapsulated nanoparticles compared to soluble active agents. Another indicator of success is the reduction in granuloma formation in infected animals measured by histologic scoring of tissues from infected animals. Success is evident by increasing killing of these strains in broth culture or intracellular viability assays measured by CFU enumeration or FACS-% viable analysis.

Stability of polyanhydride particles can be tuned by adjusting the type and relative percentages of co-polymers used for particle construction. Particle stability can be affected by the chemical and physical properties of the active agents to be encapsulated. Administration of the PANs can achieve sustained release equivalent to therapeutic concentrations over a minimum of 7 days, without exceeding levels associated with cytotoxicity. The PANs have in vitro and in vivo efficacy against multiple filarial and helminth (parasitic worm) species.

In vitro antimicrobial efficacy can be assessed using standard anti-microbial approaches of limiting dilution and growth inhibition to monitor potency differences via bacterial viability staining coupled with enumerating worm viability and motility. A mouse model for chronic granulomatous disease can be used to assess nanoparticle formulations for their ability to reduce the number of Wuchereria, Brugia spp. and O. volvulus from chronically infected animals. An additional benefit is a reduction in granuloma formation and tissue destruction.

| NTD - Filarial Pathogens | | Transmission cycle: |
|---|---|---|
| Lymphatic filariasis (LF). | Elephantiasis | Mosquitos → filarial parasites injected in |
| (dual therapy-direct injection- | | children (replicates in lymph)→years/decades |
| overlapping endemic disease | | disease to develop. |
| therapies) | | Male and female worms in lymphatics |
| Wuchereria bancrofti | | MDA w/single dose of 2 drugs albendazole |
| Brugia malayi | | (400 mg) + either ivermectin (150 mcg/kg) or |
| Brugia timori | | diethylcarbamazine citrate (DEC) (6 mg/kg) |
| | | (kill blood microfilaria and most adult worms) |
| Onchocerciasis. | River | Blackfly→filarial parasites injected in children |
| (dual therapy-eye drop and | Blindness | (replicates in lymph)→multiple exposures |
| depot of ivermectin); | | →microfilariae death is toxic to host in skin. |
| Intradermal patch; | | Microfilaria move through subcutaneous |
| Onchocerca volvulus | | layers in skin (access eye as well); |
| | | Ivermectin or moxidectin. |
| Soil-transmitted helminthes | Ascariasis | Eggs from human feces and into soil → eggs |
| (STH); | (roundworm); | hatch in 3 weeks and larvae are infectious → |
| Intradermal patch (prevention); | Trichuriasis | penetrate skin but DO NOT replicate in host |
| Intranasal for gut and systemic | (whipworm); | → repeated exposures. |
| benefits; | Hookworm | WHO rec. periodic deworming once annually; |
| Direct Gastric delivery. | | albendazole (400 mg) or mebendazole (500 mg). |
| Ascaris lumbricoides | | |
| Trichuris trichiura | | |
| Necator americanus/ | | |
| Ancylostoma duedenale | | |

### Example 5

### Pharmaceutical Dosage Forms

The following formulations illustrate representative pharmaceutical dosage forms that may be used for the therapeutic or prophylactic administration of a PANS composition described herein (hereinafter referred to as ‘Composition X’):

| | mg/tablet |
|---|---|
| (i) Tablet 1 | |
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| | mg/mL |
|---|---|
| (iv) Injection 1 (1 mg/mL) | |
| ‘Composition X’ | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | |
| ‘Composition X’ | 10.0 |
| Monobasic sodium phosphate | 0.3 |
| Dibasic sodium phosphate | 1.1 |
| Polyethylene glycol 400 | 200.0 |
| 01N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, these embodiments and examples are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method to kill a parasite or inhibit the preproduction of parasites comprising:
contacting a parasite with, or administering to the host of a parasite, an effective amount of a composition comprising polyanhydride nanoparticles, wherein the polyanhydride nanoparticles comprise:
(a) polyanhydride polymers in the form of a nanoparticle and (b) a combination of two or more different active agents located in the interior of the nanoparticle, wherein the nanoparticle is substantially spherical in shape and has an average diameter of about 100 nm to about 900 nm;
wherein the polyanhydride polymers comprise anhydride copolymers of 1,ω-bis(carboxy)(C₂-C₁₀)alkane units and 1,ω-bis(carboxyphenoxy)(C₂-C₁₀)alkane units;
wherein one active agent is an antiparasitic agent and a second active agent is an antibiotic agent; and
wherein the nanoparticles degrade by surface erosion in the presence of the parasite over a period of time to release the active agents from the interior of the nanoparticles, thereby killing the parasite or inhibiting the reproduction of the parasite.

2. The method of claim 1 wherein the 1,ω-bis(carboxy-phenoxy)(C₂-C₁₀)alkane is a 1,ω-bis(carboxy-phenoxy)(C₄-C₈)alkane.

3. The method of claim 2 wherein the 1,ω-bis(carboxy-phenoxy)(C₂-C₁₀)alkane comprises 1,6-bis-(p-carboxyphenoxy)hexane (CPH) anhydrides.

4. The method of claim 1 wherein the 1,ω-bis(carboxy)(C₂-C₁₀)alkane comprises sebacic anhydrides (SA).

5. The method of claim 1 wherein the 1,ω-bis(carboxy)(C₂-C₁₀)alkane is sebacic anhydride (SA) and the 1,ω-bis(carboxyphenoxy)(C₂-C₁₀)alkane is 1,6-bis-(p-carboxyphenoxy)hexane (CPH).

6. The method of claim 1 wherein the ratio of 1,ω-bis(carboxy)(C₂-C₁₀)alkane units to 1,ω-bis(carboxyphenoxy)(C₂-C₁₀)alkane in the nanoparticle is about 90:10 to about 70:30.

7. The method of claim 1 wherein the 1,ω-bis(carboxy)(C₂-C₁₀)alkane is sebacic anhydride (SA) and the 1,ω-bis(carboxyphenoxy)(C₂-C₁₀)alkane is 1,6-bis-(p-carboxyphenoxy)hexane (CPH) and the SA:CPH ratio is about 90:10 to about 70:30.

8. The method of claim 1 wherein the antiparasitic agent is ivermectin, abamectin, albendazole, amphotericin B, artemisinin, auranofin, chloroquine, diethylcarbamazine, eflornithine, emetine, halofantrine, mebendazole, mefloquine, metronidazole, miltofosine, moxidectin, piperazine, praziquantel, primaquine, proguanil, pyrantel pamoate, quinine, quinolones, rapamycin, spiramycin, suramin, thiabendazole, or tinidazole.

9. The method of claim 1 wherein the antibiotic agent is amikacin, bacitracin, carbapenem, ceftiofur, chloramphenicols, ciprofloxacin, clindamycin, cycloserine, doxycycline, erythromycin, ethambutol, fluoroquinolones, gentamicin, isoniazid, rifampin, streptogramin, streptomycin, tetracycline, vancomycin, or a combination thereof.

10. The method of claim 1 wherein the antiparasitic agent is ivermectin and the antibiotic agent is doxycycline.

11. The method of claim 1 wherein the antiparasitic agent comprises the combination of diethylcarbamazine and albendazole, and the antibiotic agent is doxycycline.

12. The method of claim 1 wherein the polyanhydride nanoparticle is capable of penetrating the surface (cuticle) of a parasitic worm.

13. The method of claim 1 wherein the polyanhydride nanoparticles kill parasites in less than half the time required for corresponding non-encapsulated active agents at the same concentration of total active agents.

14. The method of claim 1 wherein the parasitic infection is lymphatic filariasis (Elephantiasis).

15. The method of claim 1 wherein the parasitic infection is river blindness (Onchocerciasis).

16. The method of claim 1 wherein the parasitic infection is caused by Brugia malayi or Brugia pahangi.

17. A method to deliver active agents to a mammal infected with parasites comprising:
administering to a mammal infected by parasites an effective amount of a composition that includes polyanhydride nanoparticles and a combination of an antiparasitic agent and an antibiotic agent;
wherein the polyanhydride nanoparticles comprise copolymers of (a) 1,6-bis-(p-carboxyphenoxy)hexane (CPH) anhydride and sebacic anhydride (SA) in a ratio of about 10:90 to about 30:70; or (b) 1,8-bis(carboxyphenoxy)-3,6-dioxaoctane (CPTEG) anhydride and 1,6-bis-(p-carboxyphenoxy)hexane (CPH) anhydride in a ratio of about 10:90 to about 30:70; the nanoparticles are substantially spherical in shape, and have an average diameter of about 100 nm to about 900 nm;
the copolymers of the polyanhydride particles form a matrix around the antiparasitic agent and the antibiotic agent within the particles; and
the nanoparticles accumulate in the parasites in the mammal and degrade by surface erosion over a period of time to release the antiparasitic agent and the antibiotic agent, thereby delivering the agents to the parasites and killing or inhibiting the growth of the parasites.

18. The method of claim 17 wherein the antiparasitic agent is ivermectin or the combination of diethylcarbamazine and albendazole, and the antibiotic agent is doxycycline.

19. A polyanhydride nanoparticle comprising:
polyanhydride polymers in the form of a nanoparticle and a combination of two or more different active agents located in the interior of the nanoparticle, wherein the nanoparticle is substantially spherical in shape and has an average diameter of about 100 nm to about 900 nm;
wherein the polyanhydride polymers comprise anhydride copolymers of 1,ω-bis(carboxy)(C₂-C₁₀)alkane units and 1,ω-bis(carboxyphenoxy)(C₂-C₁₀)alkane units; and
wherein one of the active agents is an antiparasitic agent and a second active agent is an antibiotic agent.

20. The polyanhydride nanoparticle of claim 19 wherein the antiparasitic agent is ivermectin or the combination of diethylcarbamazine and albendazole, and the antibiotic agent is doxycycline.
